(19) United States
(12) Patent Application Publication
(10) Pub. No.: US 2007/0082026 A1
(43) Pub. Date: Apr. 12, 2007

(54) Compositions and methods for reducing food intake and controlling weight

(76) Inventors: William Ronald Aimutis JR., Blaine, MN (US); Janet Deihl, Doylestown, PA (US); Teresa Marie Paeschke, Minneapolis, MN (US); Timothy E. Tracy, Pennington, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/245,763

(22) Filed: Oct. 7, 2005

(51) Int. Cl. A61K 47/00; A61K 33/42

(52) U.S. Cl. 424/439; 424/602

(57) ABSTRACT

A formed food or other ingestible composition having at least one soluble anionic fiber and at least one multivalent cation wherein the combination of fiber and cation increases viscosity of digesta. Methods of weight management including inducing satiety, reducing caloric intake, weight loss and improving weight loss, using the formed food or ingestible compositions having at least one soluble anionic fiber and at least one multivalent cation.

### CROSS-REFERENCE TO RELATED APPLICATIONS

This case is related to U.S. patent application Ser. No. ______, entitled “METHODS FOR REDUCING CALORIE INTAKE” (docket number MSP5039), U.S. patent application Ser. No. ______, entitled “COMPOSITIONS AND METHODS FOR INDUCING SATIETY AND REDUCING CALORIC INTAKE” (docket number MSP5040); U.S. patent application Ser. No. ______, entitled “METHODS FOR ACHIEVING AND MAINTAINING WEIGHT LOSS” (docket number MSP5041); U.S. patent application Ser. No. ______, entitled “METHODS FOR REDUCING WEIGHT” (docket number MSP5042); U.S. patent application Ser. No. ______, entitled “COMPOSITIONS AND METHODS FOR REDUCING FOOD INTAKE AND CONTROLLING WEIGHT” (docket number MSP5043); U.S. patent application Ser. No. ______, entitled “COMPOSITIONS AND METHODS FOR REDUCING FOOD INTAKE AND CONTROLLING WEIGHT” (docket number MSP5044); U.S. patent application Ser. No. ______, entitled “METHODS FOR WEIGHT MANAGEMENT” (docket number MSP5045); U.S. patent application Ser. No. ______, entitled “METHODS FOR INDUCING SATIETY, REDUCING FOOD INTAKE AND REDUCING WEIGHT” (docket number MSP5046); U.S. patent application Ser. No. ______, entitled “COMPOSITIONS AND METHODS FOR REDUCING FOOD INTAKE AND CONTROLLING WEIGHT (docket number MSP5047); U.S. patent application Ser. No. ______, entitled “FIBER SATIETY COMPOSITIONS” (docket number 10790-056001); and U.S. patent application Ser. No. ______, entitled “FIBER SATIETY COMPOSITIONS” (docket number 10790-056002), each filed concurrently herewith on Oct. 7, 2005.

### FIELD OF THE INVENTION

The present invention is directed to formed foods that include at least one soluble anionic fiber and at least one multivalent cation and methods for inducing satiety in an animal, reducing caloric intake in an animal, reducing weight in an animal, and improving weight reduction in an animal using the ingestible compositions.

### BACKGROUND OF THE INVENTION

Diabetes and obesity are common ailments in the United States and other Western cultures. A study by researchers at RTI International and the Centers for Disease Control estimated that U.S. obesity-attributable medical expenditures reached $75 billion in 2003. Obesity has been shown to promote many chronic diseases, including type 2 diabetes, cardiovascular disease, several types of cancer, and gallbladder disease.

Adequate dietary intake of soluble fiber has been associated with a number of health benefits, including decreased blood cholesterol levels, improved glycemic control, and the induction of satiety and satiation in individuals. Consumers have been resistant to increasing soluble fiber amounts in their diet due to the negative organoleptic characteristics, such as, sliminess, excessive viscosity, excessive dryness and poor flavor, that are associated with food products that include soluble fiber.

What is needed is a stable, organoleptically acceptable food product that delivers a cation and at least one soluble anionic fiber where the cation will react with the anionic fiber to create a viscous material in vivo, but do not react in vitro over the shelf life of the product.

### SUMMARY OF THE INVENTION

The present invention solves the above needs by providing a formed food comprising, consisting of, and/or consisting essentially of an effective amount of at least one soluble anionic fiber and an effective amount of at least one multivalent cation wherein the combination of fiber and cation increases viscosity of digesta.

Another embodiment of the present invention is an ingestible composition comprising consisting of, and/or consisting essentially of a solid phase comprising, consisting of, and/or consisting essentially of at least one soluble anionic fiber in a total amount of from about 0.5 g to about 10 g per serving and a fluid phase in intimate contact with the solid phase, the fluid phase comprising, consisting of, and/or consisting essentially of calcium in an amount of from about 50 to about 300 mg of elemental calcium per serving.

A further embodiment of the present invention is directed to a method for inducing satiety in an animal, the method comprising, consisting of, and/or consisting essentially of the step of orally administering to the animal a serving of a formed food comprising at least one soluble anionic fiber and at least one multivalent cation.

Another embodiment of the present invention is a method for inducing satiety in an animal, the method comprising, consisting of, and/or consisting essentially of the step of orally administering to the animal a serving of a food comprising, consisting of, and/or consisting essentially of a formed solid phase comprising at least one soluble anionic fiber in a total amount of from about 0.5 g to about 10 g per serving and a fluid phase in intimate contact with the formed solid phase, the fluid phase comprising calcium in an amount of from about 50 to about 500 mg of elemental calcium per serving.

A still further embodiment of the present invention is a method for reducing caloric intake in an animal, the method comprising, consisting of, and/or consisting essentially of the step of orally administering to the animal a serving of a formed food comprising consisting of, and/or consisting essentially of at least one soluble anionic fiber and at least one multivalent cation.

An additional embodiment of the present invention is a method for reducing caloric intake in an animal, the method comprising, consisting of, and/or consisting essentially of the step of orally administering to the animal a serving of an food comprising, consisting of, and/or consisting essentially of a formed solid phase comprising, consisting of, and/or consisting essentially of at least one soluble anionic fiber in a total amount of from about 0.5 g to about 10 g per serving and a fluid phase in intimate contact with the formed solid phase, the fluid phase comprising, consisting of, and/or consisting essentially of calcium in an amount of from about 50 to about 300 mg of elemental calcium per serving.

A further additional embodiment of the present invention is a method for reducing weight in an animal, the method comprising, consisting of, and/or consisting essentially of the step of orally administering to the animal a serving of a formed food comprising, consisting of, and/or consisting essentially of at least one soluble anionic fiber and at least one multivalent cation.

Another embodiment of the present invention is a method for reducing weight in an animal, the method comprising, consisting of, and/or consisting essentially of the step of orally administering to the animal a serving of an ingestible composition comprising, consisting of, and/or consisting essentially of a formed solid phase comprising, consisting of, and/or consisting essentially of at least one soluble anionic fiber in a total amount of from about 0.5 g to about 10 g per serving and a fluid phase in intimate contact with the formed solid phase, the fluid phase comprising, consisting of, and/or consisting essentially of calcium in an amount of from about 50 to about 300 mg of elemental calcium per serving.

A further embodiment of the present invention is a method for improving weight reduction by at least 5% in an animal, the method comprising, consisting of, and/or consisting essentially of the step of orally administering to the animal a serving of a formed food comprising, consisting of, and/or consisting essentially of at least one soluble anionic fiber and a multivalent cation, wherein the weight reduction improvement is measured after four months of daily administration of the ingestible composition.

A still further embodiment of the present invention is a method for improving weight reduction by at least 5% in an animal, the method comprising, consisting of, and/or consisting essentially of the step of orally administering to the animal an ingestible composition comprising, consisting of, and/or consisting essentially of a formed solid phase comprising, consisting of, and/or consisting essentially of at least one soluble anionic fiber in a total amount of from about 0.5 g to about 10 g per serving and a fluid phase in intimate contact with the formed solid phase, the fluid phase comprising calcium in an amount of from about 50 to about 300 mg of elemental calcium per serving, wherein the weight reduction improvement is measured after four months of daily administration of the ingestible composition.

An additional embodiment of the present invention is an ingestible composition comprising, consisting of, and/or consisting essentially of a formed solid phase comprising, consisting of, and/or consisting essentially of at least one soluble anionic fiber in a total amount of from about 0.5 g to about 10 g per serving and a fluid phase in intimate contact with the baked phase, the fluid phase comprising, consisting of, and/or consisting essentially of calcium in an amount of from about 50 to about 300 mg of elemental calcium per serving.

### BRIEF DESCRIPTION OF DRAWINGS

FIG. 1 is a graph depicting the effects of an embodiment of the present invention on intestinal viscosity.

### DETAILED DESCRIPTION OF THE INVENTION

As used herein, unless indicated otherwise, the terms “alginate,” “pectin,” “carrageenan,” “polygeenan,” or “gellan” refers to all forms (e.g., protonated or salt forms, such as sodium, potassium, and ammonium salt forms and having varying average molecular weight ranges) of the soluble anionic fiber type.

As used herein, unless indicated otherwise, the term “alginic acid” includes not only the material in protonated form but also the related salts of alginate, including, but not limited, to sodium, potassium, and ammonium alginate.

As used herein, unless indicated otherwise, the term “protected” means that the source has been treated in such a way, as illustrated below, to delay (e.g., until during or after ingestion or until a certain pH range has been reached) reaction of the at least one multivalent cation with the soluble anionic fiber as compared to an unprotected multivalent cation.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

As used herein, a recitation of a range of values is merely intended to serve as a shorthand method of referring individually to each separate value falling within the range, and each separate value is incorporated into the specification as if it were individually recited herein.

Soluble Anionic Fiber

Any soluble anionic fiber should be acceptable for the purposes of this invention. Suitable soluble anionic fibers include alginate, pectin, gellan, soluble fibers that contain carboxylate substituents, carrageenan, polygeenan, and marine algae-derived polymers that contain sulfate substituents.

Also included within the scope of soluble anionic fibers are other plant derived and synthetic or semisynthetic polymers that contain sufficient carboxylate, sulfate, or other anionic moieties to undergo gelling in the presence of sufficient levels of multivalent cation.

At least one source of soluble anionic fiber may be used in these compositions, and the at least one source of soluble anionic fiber may be combined with at least one source of soluble fiber that is uncharged at neutral pH. Thus, in certain cases, two or more soluble anionic fibers types are included, such as, alginate and pectin, alginate and gellan, or pectin and gellan. In other cases, only one type of soluble anionic fiber is used, such as only alginate, only pectin, only carrageenan, or only gellan.

Soluble anionic fibers are commercially available, e.g., from ISP (Wayne, NJ), TIC Gums, and CP Kelco.

An alginate can be a high guluronic acid alginate. For example, in certain cases, an alginate can exhibit a higher than 1:1 ratio of guluronic to mannuronic acids, such as in the range from about 1.2:1 to about 1.8:1, e.g., about 1.3:1, about 1.4:1, about 1.5:1, about 1.6:1, or about 1.7:1 or any value therebetween. Examples of high guluronic alginates (e.g., having a higher than 1:1 g:m ratios) include Manugel LBA, Manugel GHB, and Manugel DBP, which each have a g:m ratio of about 1.5.

While not being bound by theory, it is believed that high guluronic alginates can cross-link through multivalent cations, e.g., calcium ions, to form gels at the low pH regimes in the stomach. High guluronic alginates are also believed to electrostatically associate with pectins and/or gellans at low pHs, leading to gellation. In such cases, it may be useful to delay the introduction of multivalent cations until after formation of the mixed alginate/pectin or alginate/gellan gel, as multivalent cationic cross-links may stabilize the mixed gel after formation.

In other cases, an alginate can exhibit a ratio of guluronic to mannuronic acids (g:m ratio) of less than about 1:1, e.g., about 0.8:1 to about 0.4:1, such as about 0.5:1, about 0.6:1, or about 0.7:1 or any value therebetween. Keltone LV and Keltone HV are examples of high-mannuronic acids (e.g., having a g:m ratio of less than 1:1) having g:m ratios ranging from about 0.6:1 to about 0.7:1.

Methods for measuring the ratio of guluronic acids to mannuronic acids are known by those having ordinary skill in the art.

An alginate can exhibit any number average molecular weight range, such as a high molecular weight range (about 2.05×10⁵ to about 3×10⁵ Daltons or any value therebetween; examples include Manugel DPB, Keltone HV, and TIC 900 Alginate); a medium molecular weight range (about 1.38×10⁵ to about 2×10⁵ Daltons or any value therebetween; examples include Manugel GHB); or a low molecular weight range (2×10⁴ to about 1.35×10⁵ Daltons or any value therebetween; examples include Manugel LBA and Manugel LBB). Number average molecular weights can be determined by those having ordinary skill in the art, e.g., using size exclusion chromatography (SEC) combined with refractive index (RI) and multi-angle laser light scattering (MALLS).

In certain embodiments of a formed food product, a low molecular weight alginate can be used (e.g., Manugel LBA), while in other cases a mixture of low molecular weight (e.g., Manugel LBA) and high molecular weight (e.g., Manugel DPB, Keltone HV) alginates can be used. In other cases, a mixture of low molecular weight (e.g., Manugel LBA) and medium molecular weight (e.g., Manugel GHB) alginates can be used. In yet other cases, one or more high molecular weight alginates can be used (e.g., Keltone HV, Manugel DPB).

A pectin can be a high-methoxy pectin (e.g., having greater than 50% esterified carboxylates), such as ISP HM70LV and CP Kelco USPL200. A pectin can exhibit any number average molecular weight range, including a low molecular weight range (about 1×10⁵ to about 1.20×10⁵ Daltons, e.g., CP Kelco USPL200), medium molecular weight range (about 1.25×10⁵ to about 1.45×10⁵, e.g., ISP HM70LV), or high molecular weight range (about 1.50×10⁵ to about 1.80×10⁵, e.g., TIC HM Pectin). In certain cases, a high-methoxy pectin can be obtained from pulp, e.g., as a by-product of orange juice processing.

A gellan soluble anionic fiber can also be used. Gellan fibers form strong gels at lower concentrations than alginates and/or pectins, and can cross-link with multivalent cation cations. For example, gellan can form gels with magnesium and calcium. Gellans for use in the invention include Kelcogel, available commercially from CP Kelco.

A range of fiber intake in the compositions of this invention may be about about 0.25 g to about 5 g per serving, more preferably about 0.5 to about 3 g per serving, and most preferably about 1.0 to about 2.0 g per serving.

Fiber blends as described herein can also be used in the preparation of a solid ingestible composition like an extruded food product where the fiber blend is a source of the soluble anionic fiber. A useful fiber blend can include an alginate soluble anionic fiber and a pectin soluble anionic fiber. A ratio of total alginate to total pectin in a blend can be from about 8:1 to about 5:1, or any value therebetween, such as about 7:1, about 6.5:1, about 6.2:1, or about 6.15:1. A ratio of a medium molecular weight alginate to a low molecular weight alginate can range from about 0.65:1 to about 2:1, or any value therebetween.

An alginate soluble anionic fiber in a blend may be a mixture of two or more alginate forms, e.g., a medium and low molecular weight alginate. In certain cases, a ratio of a medium molecular weight alginate to a low molecular weight alginate is about 0.8:1 to about 0.9:1. The fiber blend combining low and medium molecular weight alginates with high methoxy pectin has been tested at about 0 to about 3 grams. A preferred range for both may be about 1 to about 2 grams.

The at least one soluble anionic fiber may be treated before, during, or after incorporation into an ingestible composition. For example, the at least one soluble anionic fiber can be processed, e.g., extruded, roll-dried, freeze-dried, dry blended, roll-blended, agglomerated, coated, or spray-dried.

For solid forms, a variety of formed food products can be prepared by methods known to those having ordinary skill in the art, e.g., extruding, molding, pressing, wire-cutting, and the like. For example, a single or double screw extruder can be used. Typically, a feeder meters in the raw ingredients to a barrel that includes the screw(s). The screw(s) conveys the raw material through the die that shapes the final product. Extrusion can take place under high temperatures and pressures or can be a non-cooking, forming process. Extruders are commercially available, e.g., from Buhler, Germany. Extrusion can be cold or hot extrusion.

Other processing methods are known to those having skilled in the art.

The amount of the at least one soluble anionic fiber included can vary, and will depend on the type of ingestible composition and the type of soluble anionic fiber used. For example, typically an ingestible composition, e.g., formed food, will include from about 0.5 g to about 10 g total soluble anionic fiber per serving or any value therebetween. In certain cases, a formed food product may include an soluble anionic fiber at a total amount from about 22% to about 40% by weight of the formed food product or any value therebetween. In other cases, a formed food product may include an soluble anionic fiber in a total amount of from about 4% to about 15% or any value therebetween, such as when only gellan is used. In yet other cases, a formed food product can include an soluble anionic fiber at a total amount of from about 18% to about 25% by weight, for example, when combinations of gellan and alginate or gellan and pectin are used.

In addition to the at least one soluble anionic fiber, a solid ingestible composition can include ingredients that may be treated in a similar manner as the at least one soluble anionic fiber. For example, such ingredient can be co-extruded with the soluble anionic fiber, co-processed with the soluble anionic fiber, or co-spray-dried with the soluble anionic fiber. Such treatment can help to reduce sliminess of the ingestible composition in the mouth and to aid in hydration and gellation of the fibers in the stomach and/or small intestine. Without being bound by any theory, it is believed that co-treatment of the soluble anionic fiber(s) with such ingredient prevents early gellation and hydration of the fibers in the mouth, leading to sliminess and unpalatability. In addition, co-treatment may delay hydration and subsequent gellation of the soluble anionic fibers (either with other soluble anionic fibers or with multivalent cations) until the ingestible composition reaches the stomach and/or small intestine, providing for the induction of satiety and/or satiation.

Additional ingredients can be hydrophilic in nature, such as, starch, protein, maltodextrin, and inulin. Other additional ingredients can be insoluble in water (e.g., cocoa solids, corn fiber) and/or fat soluble (vegetable oil), or can be flavor modifiers, such as, sucralose. For example, a formed food product can include from about 5 to about 80% of a cereal ingredient, such as, about 40% to about 68% of a cereal ingredient. A cereal ingredient can be rice, corn, wheat, sorghum, oat, or barley grains, flours, or meals. Thus, an extruded food product can include about 40% to about 50%, about 50% to about 58%, about 52% to about 57%, or about 52%, about 53%, about 54%, about 55%, about 56%, or about 56.5% of a cereal ingredient. In one embodiment, about 56.5% of rice flour is included.

A formed food may also include a protein source. A protein source may be included in the composition. For example, an extruded food product can include a protein source at about 2% to about 20% by weight, such as about 3% to about 8%, about 3% to about 5%, about 4% to about 7%, about 4% to about 6%, about 5% to about 7%, about 5% to about 15%, about 10% to about 18%, about 15% to about 20%, or about 8% to about 18% by weight. A protein can be any known to those having ordinary skill in the art, e.g., rice, milk, egg, wheat, whey, soy, gluten, or soy flour. In some cases, a protein source can be a concentrate or isolate form.

Multivalent Cation

The compositions and associated methods of this invention include a source of at least one multivalent, e.g., divalent cation in an amount sufficient to cause an increase in viscosity of the digesta of an animal. A source of at least one multivalent cation may be incorporated into an ingestible composition provided herein, or can consumed as a separate food article either before, after, or simultaneously with an ingestible composition.

Any multivalent cation may be used in the present invention, e.g., divalent, trivalent, and the like. Multivalent cations useful in this invention include, calcium, magnesium, aluminum, manganese, their salts and mixtures thereof. Salts of the multivalent cations may be organic acid salts that include formate, fumarate, acetate, propionate, butyrate, caprylate, valerate, lactate, citrate, malate and gluconate. Also included are highly soluble inorganic salts such as chlorides or other halide salts.

In certain compositions, one or more particular multivalent cations may be used with certain soluble anionic fibers, depending on the composition and gel strength desired. For example, for ingestible alginate compositions, calcium may be used to promote gellation. For gellan compositions, one or more of calcium and magnesium may be used.

The at least one multivalent cation can be unable to, or be limited in its ability to, react with the at least one soluble anionic fiber in the ingestible composition until during or after ingestion. For example, physical separation of the at least one multivalent cation from the at least one soluble anionic fiber, e.g., as a separate food article or in a separate matrix of the ingestible composition from the at least one soluble anionic fiber, can be used to limit at least one multivalent cation's ability to react. In other cases, the at least one multivalent cation is limited in its ability to react with the at least one soluble anionic fiber by protecting the source of at least one multivalent cation until during or after ingestion. Thus, the at least one multivalent cation, such as, a protected multivalent cation, can be included in the ingestible composition or can be included as a separate food article composition, e.g., for separate ingestion either before, during, or after ingestion of an ingestible composition.

Typically, a separate food article containing the source of at least one multivalent cation would be consumed in an about four hour time window flanking the ingestion of an ingestible composition containing the at least one soluble anionic fiber. In certain cases, the window may be about three hours, or about two hours, or about one hour. In other cases, the separate food article may be consumed immediately before or immediately after ingestion of an ingestible composition, e.g., within about fifteen minutes, such as within about 10 minutes., about 5 minutes, or about 2 minutes. In other cases, a separate food article containing at least one multivalent cation can be ingested simultaneously with an ingestible composition containing the at least one soluble anionic fiber, e.g., a snack chip composition where some chips include at least one multivalent cation and some chips include the at least one soluble anionic fiber.

In one embodiment, at least one multivalent cation can be included in an ingestible composition in a different food matrix from a matrix containing an soluble anionic fiber. For example, a source of at least one multivalent cation, such as a calcium salt, can be included in a separate matrix of a solid ingestible composition from the matrix containing the at least one soluble anionic fibers. Thus, means for physical separation of an soluble anionic fiber (e.g., within a snack bar or other extruded food product) from a source of at least one multivalent cation are also contemplated, such as by including the source of at least one multivalent cation in a matrix such as a frosting, water and fat based icing, coating, decorative topping, drizzle, chip, chunk, swirl, filling, or interior layer. In one embodiment, a source of at least one multivalent cation, such as a protected multivalent cation source, can be included in a snack bar matrix that also contains an extruded crispy matrix that contains the soluble anionic fiber. In such a case, the source of at least one multivalent cation is in a separate matrix than the extruded crispy matrix containing the soluble anionic fiber. In another embodiment, a source of at least one multivalent cation can be included in a gel layer or phase, e.g., a jelly or jam.

One multivalent cation source is multivalent cation salts. Typically, a multivalent cation salt can be selected from the following salts: citrate, tartrate, malate, formate, lactate, gluconate, phosphate, carbonate, sulfate, chloride, acetate, propionate, butyrate, caprylate, valerate, fumarate, adipate, and succinate. In certain cases, a multivalent cation salt is a calcium salt. A calcium salt can have a solubility of >1% w/vol in water at pH 7 at 20° C. A calcium salt can be, without limitation, calcium citrate, calcium tartrate, calcium malate, calcium lactate, calcium gluconate, dicalcium phosphate dihydrate, anhydrous calcium diphosphate, calcium citrate malate, dicalcium phosphate anhydrous, calcium carbonate, calcium sulfate dihydrate, calcium sulfate anhydrous, calcium chloride, calcium acetate monohydrate, monocalcium phosphate monohydrate, and monocalcium phosphate anhydrous.

The source of at least one multivalent cation can be a protected source.

A number of methods can be used to protect a source of at least one multivalent cation. For example, microparticles or nanoparticles having double or multiple emulsions, such as water/oil/water (“w/o/w”) or oil/water/oil (“o/w/o”) emulsions, of at least one multivalent cation and an soluble anionic fiber can be used. In one embodiment, a calcium alginate microparticle or nanoparticle is used. For example, a calcium chloride solution can be emulsified in oil, which emulsion can then be dispersed in a continuous water phase containing the anionic alginate soluble fiber. When the emulsion breaks in the stomach, the calcium can react with the alginate to form a gel.

A microparticle can have a size from about 1 to about 15 μM (e.g., about 5 to about 10 μM, or about 3 to about 8 μM). A nanoparticle can have a size of about 11 to about 85 nm (e.g., about 15 to about 50 nm, about 30 to about 80 nm, or about 50 to about 75 nm). The preparation of multiple or double emulsions, including the choice of surfactants and lipids, is known to those having ordinary skill in the art.

In another embodiment, nanoparticles of calcium alginate are formed by preparing nanodroplet w/o microemulsions of CaCl₂ in a solvent and nanodroplet w/o microemulsions of alginate in the same solvent. When the two microemulsions are mixed, nanoparticles of calcium alginate are formed. The particles can be collected and dispersed, e.g., in a fluid ingestible composition. As the particle size is small (<100 nm), the particles stay dispersed (e.g., by Brownian motion), or can be stabilized with a food grade surfactant. Upon ingestion, the particles aggregate and gel.

In other embodiments, a liposome containing a source of at least one multivalent cation can be included in an ingestible composition. For example, a calcium-containing liposome can be used. The preparation of liposomes containing multivalent cations is well known to those having ordinary skill in the art; see ACS Symposium Series, 1998 709:203-211; Chem. Mater. 1998 (109-116). Cochelates can also be used, e.g., as described in U.S. Pat. No. 6,592,894 and U.S. Pat. No. 6,153, 217. The creation of cochelates using multivalent cations such as calcium can protect the multivalent cations from reacting with the soluble anionic fiber within the aqueous phase of an ingestible composition, e.g., by wrapping the multivalent cations in a hydrophobic lipid layer, thus delaying reaction with the fiber until digestion of the protective lipids in the stomach and/or small intestine via the action of lipases.

In certain cases, a multivalent cation-containing carbohydrate glass can be used, such as a calcium containing carbohydrate glass. A carbohydrate glass can be formed from any carbohydrate such as, without limitation, sucrose, trehalose, inulin, maltodextrin, corn syrup, fructose, dextrose, and other mono-, di-, or oligo-saccharides using methods known to those having ordinary skill in the art; see, e.g., WO 02/05667. A carbohydrate glass can be used, e.g., in a coating or within a food matrix.

Proteins can also be used to encapsultate the multivalent cation.

Ingestible Compositions

Compositions of the present invention include formed foods and ingestible compositions having a solid phase and a fluid phase.

Compositions of the present invention include bread, cracker, bar, mini-bars, cookie, confectioneries, e.g., nougats, toffees, fudge, caramels, hard candy enrobed soft core, icings, frostings, fruit leathers, muffins, cookies, brownies, cereals, chips, snack foods, bagels, chews, crispies, and nougats, pudding, jelly, and jam. Compositions of the present invention can have densities from low to high.

Fluids

A fluid phase can be useful for, among other things, delivering about 1 g, 2 g, 3 g, 4 g, 5 g, 6 g, 7 g, 8 g, or 9 g of at least one soluble anionic fiber per serving.

A fluid phase may include an alginate soluble anionic fiber and/or a pectin soluble anionic fiber. In certain cases, an alginate soluble anionic fiber and a pectin soluble anionic fiber are used. A fiber blend as described herein can be used to provide the alginate soluble anionic fiber and/or the pectin soluble anionic fiber. An alginate and pectin can be any type and in any form, as described previously. For example, an alginate can be a high, medium, or low molecular weight range alginate, and a pectin can be a high-methoxy pectin. Also as indicated previously, two or more alginate forms can be used, such as a high molecular weight and a low molecular weight alginate, or two high molecular weight alginates, or two low molecular weight alginates, or a low and a medium molecular weight alginate, etc. For example, Manugel GHB alginate and/or Manugel LBA alginate can be used. In other cases, Manugel DPB can be used. Genu Pectin, USPL200 (a high-methoxy pectin) can be used as a pectin. In certain cases, potassium salt forms of an soluble anionic fiber can be used, e.g., to reduce the sodium content of an ingestible composition.

A fluid phase may include alginate and/or pectin in a total amount of about 0.3% to about 5% by weight, or any value therebetween, e.g., about 1.25% to about 1.9%; about 1.4% to about 1.8%; about 1.0% to about 2.2%, about 2.0% to about 4.0%, about 3.0%, about 4.0%, about 2.0%, about 1.5%, or about 1.5% to about 1.7%. Such percentages of total alginate and pectin can yield about 2 g to about 8 g of fiber per 8 oz. serving, e.g., about 3 g, about 4 g, about 5 g, about 6 g, or about 7 g fiber per 8 oz. serving. In other cases, about 4 g to about 8 g of fiber (e.g., about 5 g, about 6 g, or about 7 g) per 12 oz. serving can be targeted. In some embodiments, about 1.7% fiber by weight of a fluid ingestible composition is targeted.

In some cases, a fluid phase may include only alginate as a soluble anionic fiber. In other cases, alginate and pectin are used. A ratio of alginate to pectin (e.g., total alginate to total pectin) in a fluid ingestible composition can range from about 8:1 to about 1:8, and any ratio therebetween (e.g., alginate:pectin can be in a ratio of about 1: 1, about 1.2:1, about 1.3:1, about 1.4:1, about 1.5:1, about 1.6:1, about 1.62:1, about 1.7:1, about 1.8:1, about 1.9:1, about 2:1, about 3:1, about 4:1, about 5:1, about 5.3:1, about 5.6:1, about 5.7:1, about 5.8:1, about 5.9:1, about 6:1, about 6.1:1, about 6.5:1, about 7:1, about 7.5:1, about 7.8:1, about 2:3, about 1:4, or about 0.88:1). In cases where alginate and pectin are in a ratio of about 0.5:1 to about 2:1, it is believed that pectin and alginate electrostatically associate with one another to gel in the absence of multivalent cations; thus, while not being bound by theory, it may be useful to delay the introduction of multivalent cations until after such gel formation. In other cases, where the ratio of alginate to pectin is in the range from about 3:1 to about 8:1, it may be useful to include a multivalent cation source such as a calcium source (e.g., to crosslink the excess alginate) to aid gel formation in the stomach. In these cases, the inventors believe, while not being bound by any theory, that the lower amount of pectin protects the alginate from precipitating as alginate at the low pHs of the stomach environment, while the multivalent cation source cross-links and stabilizes the gels formed.

A fluid phase can have a pH from about 3.9 to about 4.5, e.g., about 4.0 to about 4.3 or about 4.1 to about 4.2. At these pHs, it is believed that the fluid phase are above the pKas of the alginate and pectin acidic subunits, minimizing precipitation, separation, and viscosity of the solutions. In some cases, malic, phosphoric, and citric acids can be used to acidify the compositions. In some cases, a fluid ingestible composition can have a pH of from about 5 to about 7.5. Such fluid ingestible compositions can use pH buffers known to those having ordinary skill in the art.

Sweeteners useful in the present invention can vary according to the use of the composition. Low glycemic sweeteners including trehalose, isomaltulose, aspartame, saccharine, and sucralose can be used. Sucralose can be used alone in certain formulations. The choice of sweetener will impact the overall caloric content of a composition. In certain cases, a composition can be targeted to have 40 calories/12 oz serving.

A fluid phase can demonstrate gel strengths of about 20 to about 250 grams force (e.g., about 60 to about 240, about 150 to about 240, about 20 to 30, about 20 to about 55, about 50 to 200; about 100 to 200; and about 175 to 240), as measured in a static gel strength assay. Gel strengths can be measured in the presence and absence of a multivalent cation source, such as a calcium source.

A fluid phase can exhibit a viscosity in the range of from about 15 to about 100 cPs, or any value therebetween, at a shear rate of about 10⁻⁵, e.g., about 17 to about 24; about 20 to about 25; about 50 to 100, about 25 to 75, about 20 to 80, or about 15 to about 20 cPs. Viscosity can be measured by those skilled in the art, e.g., by measuring flow curves of solutions with increasing shear rate using a double gap concentric cyclinder fixture (e.g., with a Parr Physica Rheometer).

A fluid phase can include a multivalent cation sequestrant, e.g., to prevent premature gellation of the soluble anionic fibers. A multivalent cation sequestrant can be selected from EDTA and its salts, EGTA and its salts, sodium citrate, sodium hexametaphosphate, sodium acid pyrophosphate, trisodium phosphate anhydrous, tetrasodium pyrophosphate, sodium tripolyphosphate, disodium phosphate, sodium carbonate, and potassium citrate. A multivalent cation sequestrant can be from about 0.001% to about 0.3% by weight of the ingestible composition. Thus, for example, EDTA can be used at about 0.0015%to about 0.002% by weight of the ingestible composition and sodium citrate at about 0.230% to about 0.260% (e.g., 0.250%) by weight of the ingestible composition.

A fluid phase can include a juice or juice concentrate and optional flavorants and/or colorants. Juices for use include fruit juices such as apple, grape, raspberry, blueberry, cherry, pear, orange, melon, plum, lemon, lime, kiwi, passionfruit, blackberry, peach, mango, guava, pineapple, grapefruit, and others known to those skilled in the art. Vegetable juices for use include tomato, spinach, wheatgrass, cucumber, carrot, peppers, beet, and others known to those skilled in the art.

The brix of the juice or juice concentrate can be in the range of from about 15 to about 85 degrees, such as about 25 to about 50 degrees, about 40 to about 50 degrees, about 15 to about 30 degrees, about 65 to about 75 degrees, or about 70 degrees. A fluid ingestible composition can have a final brix of about 2 to about 25 degrees, e.g., about 5, about 10, about 12, about 15, about 20, about 2.5, about 3, about 3.5, about 3.8, about 4, or about 4.5.

Flavorants can be included depending on the desired final flavor, and include flavors such as kiwi, passionfruit, pineapple, coconut, lime, creamy shake, peach, pink grapefruit, peach grapefruit, pina colada, grape, banana, chocolate, vanilla, cinnamon, apple, orange, lemon, cherry, berry, blueberry, blackberry, apple, strawberry, raspberry, melon(s), coffee, and others, available from David Michael, Givaudan, Duckworth, and other sources.

Colorants can also be included depending on the final color to be achieved, in amounts quantum satis that can be determined by one having ordinary skill in the art.

Solids

At least one soluble anionic fiber can be present in a solid ingestible composition in any form or in any mixtures of forms. A solid phase can be a processed, unprocessed, or both. Processed forms include extruded forms, spray-dried forms, roll-dried forms, or dry-blended forms. For example, a snack bar can include at least anionic soluble anionic fiber present as an extruded food product (e.g., a crispy), at least one soluble anionic fiber in an unextruded form (e.g., as part of the bar), or both.

A formed food product can be cold- or hot-extruded and can assume any type of extruded form, including without limitation, a bar, cookie, bagel, crispy, puff, curl, crunch, ball, flake, square, nugget, and snack chip. In some cases, a formed food product is in bar form, such as a snack bar or granola bar. In some cases, a formed food product is in cookie form. In other cases, a formed food product is in a form such as a crispy, puff, flake, curl, ball, crunch, nugget, chip, square, chip, or nugget. Such formed food products can be eaten as is, e.g., cookies, bars, chips, and crispies (as a breakfast cereal) or can be incorporated into a solid ingestible composition, e.g., crispies incorporated into snack bars.

A solid form may also be a lollipop or a lolly that is made of hardened, flavored sugar mounted on a stick and intended for sucking or licking. One form of lollipop has a soft-chewy filling in the center of the hardened sugar. The soft center filling may be a gum, fudge, toffee, caramel, jam, jelly or any other soft-chewy filling known in the art. The at least one multivalent cation may be in the soft-chewy center or the harnend sugar. Likewise, at least fiber may be in the soft-chewy center or the harnend sugar. A hard candy filled with a soft center filling is another embodiment of the present invention. This embodiment is similar to the lollipop, except it is not mounted on a stick. The soft-chewy filling may be in the center or swirled or layered with the hard sugar confection.

A cookie or mini-bar can include at least one soluble anionic fiber in an unformed form or in a formed (e.g., extruded) form. A snack chip can include at least one soluble anionic fiber in extruded form or in spray-dried form, or both, e.g., an extruded soluble anionic fiber-containing chip having at least one anionic soluble fiber spray-dried on the chip.

A solid ingestible composition can include optional additions such as frostings, icings, coatings, toppings, drizzles, chips, chunks, swirls, or layers. Such optional additions can include at least one multivalent cation, at least one soluble anionic fiber, or both.

Solid ingestible compositions can provide any amount from about 0.5 g to about 10 g total soluble anionic fiber per serving, e.g., about 0.5 g to about 5 g, about 1 g to about 6 g, about 3 g to about 7 g, about 5 g to about 9 g, or about 4 g to about 6 g. For example, in some cases, about 1 g, about 2 g, about 3 g, about 4 g, about 5 g, about 6 g, about 7 g, about 8 g, or about 9 g of soluble anionic fiber per serving can be provided.

A solid ingestible composition can include at least one soluble anionic fiber at a total weight percent of the ingestible composition of from about 4% to about 50% or any value therebetween. For example, a solid ingestible composition can include at least one soluble anionic fiber of from about 4% to about 10% by weight; or about 5% to about 15% by weight; or about 10% to about 20% by weight; or about 20% to about 30% by weight; or about 30% to about 40% by weight; or about 40% to about 50% by weight.

An extruded food product can be from about 0% to 100% by weight of an ingestible composition, or any value therebetween (about 1% to about 5%; about 5% to about 10%; about 10% to about 20%; about 20% to about 40%; about 30% to about 42%; about 35% to about 41%; about 37% to about 42%; about 42% to about 46%; about 30% to about 35%; about 40% to about 50%; about 50% to about 60%; about 60% to about 70%; about 70% to about 80%; about 80% to about 90%; about 90% to about 95%; about 98%; or about 99%). For example, an extruded bar, cookie, cracker, or chip can be about 80% to about 100% by weight of an ingestible composition or any value therebetween.

Alternatively, an ingestible composition can include about 30% to about 55% by weight of an extruded food product or any value therebetween, e.g., about 32%, about 33%, about 34%, about 35%, about 36%, about 37%, 3 about 8%, about 39%, about 40%, about 42%, about 45%, about 48%, about 50%, about 52%, or about 54% by weight of an extruded food product. For example, a snack bar composition can include extruded crispies in an amount of from about 32% to about 46% by weight of the snack bar.

An ingestible composition or extruded food product can include one or more of the following: cocoa, including flavonols, and oils derived from animal or vegetable sources, e.g., soybean oil, canola oil, corn oil, safflower oil, sunflower oil, etc. For example, an extruded food product can include cocoa or oils in an amount of about 3% to about 10% (e.g., about 3% to about 6%, about 4% to about 6%, about 5%, about 6%, about 7%, or about 4% to about 8%) by weight of the extruded food product.

One embodiment of the present invention is a stable two phase product having at least one soluble anionic fiber and at least one multivalent cation in the same product, but formulated so that the soluble anionic fiber and multivalent cation do not react during processing or prior to ingestion, but react following ingestion as a standard multivalent cation-anion fiber reaction. One product design includes a fluid phase center and a crisp solid phase outside the fluid phase. One embodiment places the soluble anionic fiber in the fluid phase and places the multivalent cation in the solid phase, e.g., alginate and pectin in the jam and calcium fumarate in the baked dough. This embodiment, while advantageous over more homogeneous highly gel forming compositions is less than optimal from an organoleptic standpoint. Specifically it provided a somewhat rubber-like jam phase instead of pleasant texture.

Another embodiment of the present invention addresses this issue, wherein this problem is solved by adding the soluble anionic fiber to the baked dough solid phase and the multivalent cation to the fluid jam phase. Such an embodiment provides a cookie that reduces the water activity of the fiber-containing phase, which restricts the fiber so that it is prevented from reacting with the multivalent cation in the jam. The placement of the multivalent cation into a postbake, medium water activity filler, e.g., the fluid phase, allows the cation to be formulated in the product with an acceptable organoleptic profile and an inability to react with fiber even if minor migration occurs.

The water activity of both components can be further adjusted to deliver a product with not only restrictive reaction in place but acceptable eating qualities and the right characteristics needed to for ease of manufacturing.

Types of salts useful include calcium fumarate, tricalcium phosphate, dicalcium phosphate dihydrate and calcium carbonate. The gram weight tested will vary depending on the salt type due to its characteristic calcium load. The piece weight of the product under discussion has been about 13 to about 20g, with each piece delivering about 50 to about 75 kcal.

BENEFAT® is a family of triglyceride blends made from the short and long chain fatty acids commonly present in the diet. It is the uniqueness of these fatty acids that contribute to the range's reduced calorie claim. BENEFAT® products are designed to replace conventional fats and oils in dairy, confectionery and bakery products, giving full functionality with significantly reduced energy and fat content. BENEFAT® is the Danisco trade name for SALATRIM, the abbreviation for short and long-chain triglyceride molecules. The short-chain acids (C₂-C₄) may be acetic, propionic, butyric or a combination of all three, while the long-chain fatty acid (C₁₆-C₂₂) is predominantly stearic and derived from fully hardened vegetable oil. Unlike other saturated fatty acids, stearic acid has a neutral effect on blood cholesterol. BENEFAT® is also free of trans fatty acids and highly resistant to oxidation. Compared to the 9 calories per gram of traditional fat, BENEFAT® contains just 5 calories per gram (US regulation) or 6 calories per gram (EU regulation), at the same time giving foods a similar creamy taste, texture, and mouthfeel as full-fat products. Metabolism upon consumption occurs in much the same way as with other food components.

Preferred product features include about 500 to about 1500 mg of alginate and the multivalent cation is calcium wherein about 50 to about 500mg of elemental calcium are delivered. The product has low calories between about 50 to about 100 calories and can be a cookie/mini-bar/cookie/ cereal bar with a jam filling.

The ingestible composition of the present invention can be provided in any package, such as enclosed in a wrapper or included in a container. An ingestible composition can be included in an article of manufacture. An article of manufacture that includes an ingestible composition described herein can include auxiliary items such as straws, napkins, labels, packaging, utensils, etc.

An article of manufacture can include a source of at least one multivalent cation. For example, a source of at least one multivalent cation can be provided as a fluid, e.g., as a beverage to be consumed before, during, or after ingestion of the ingestible composition. In other cases, at least one multivalent cation can be provided in a solid or gel form. For example, a source of at least one multivalent cation can be provided in, e.g., a jelly, jam, dip, swirl, filling, or pudding, to be eaten before, during, or after ingestion of the ingestible composition. Thus, in some embodiments, an article of manufacture that includes a cookie or bar solid ingestible composition can also include a dip comprising a source of at least one multivalent cation, e.g., into which to dip the cookie or bar solid ingestible composition.

Also provided are articles of manufacture that include a fluid ingestible composition. For example, a fluid ingestible composition can be provided in a container. Supplementary items such as straws, packaging, labels, etc. can also be included. Alternatively, the soluble anionic fiber may be included in a beverage and the multivalent cation may be provided inside, outside or both of a straw or stirring stick. In some cases, at least one multivalent cation, as described below, can be included in an article of manufacture. For example, an article of manufacture can include a fluid ingestible composition in one container, and a source of multivalent cations in another container. Two or more containers may be attached to one another.

Methods of Weight Management

An soluble anionic fiber (such as alginate and pectin) is orally administered concurrently with a multivalent cation source such as a water-soluble calcium salt, to reduce caloric intake, inducing satiety, reducing weight and/or improving weight reduction. Continued use of these compositions by individuals will result in a cumulative decrease in caloric consumption, which will result in weight loss or diminished weight gain. Although not wishing to be bound by theory, the inventors hypothesize that the multivalent cation calcium ions of the soluble calcium source cross link the carboxylate groups on the fiber molecules, resulting in the formation of highly viscous or gelled materials. This gelling effect increases the viscosity of the gastric and intestinal contents, slowing gastric emptying, and also slowing the rate of macro-nutrient, e.g., glucose, amino acids, fatty acids, and the like, absorption. These physiological effects prolong the period of nutrient absorption after a meal, and therefore prolong the period during which the individual experiences an absence of hunger. The increased viscosity of the gastrointestinal contents, as a result of the slowed nutrient absorption, also causes a distal shift in the location of nutrient absorption. This distal shift in absorption may trigger the so-called “ileal brake”, and the distal shift may also cause in increase in the production of satiety hormones such as GLP-1 and PYY.

Provided herein are methods employing the ingestible compositions described herein. For example, a method of inducing satiety and/or satiation in an animal is provided. The method can include administering an ingestible composition to an animal. An animal can be any animal, including a human, monkey, mouse, rat, snake, cat, dog, pig, cow, sheep, horse, bird, or horse. Administration can include providing the ingestible combination either alone or in combination with other meal items. Administration can include co-administering, either before, after, or during administration of the ingestible composition, a source of at least one multivalent cation, such as calcium or a sequestered source of calcium, as described herein. At least one multivalent cation can be administered within about a four hour time window flanking the administration of the ingestible composition. For example, a source of calcium, such as a solution of calcium lactate, can be administered to an animal immediately after the animal has ingested a fluid ingestible composition as provided herein. Satiety and/or satiation can be evaluated using consumer surveys (e.g., for humans) that can demonstrate a statistically significant measure of increased satiation and/or satiety. Alternatively, data from paired animal sets showing a statistically significant reduction in₌total caloric intake or food intake in the animals administered the ingestible compositions can be used as a measure of facilitating satiety and/or satiation.

As indicated previously, the ingestible compositions provide herein can hydrate and gel in the stomach and/or small intestine, leading to increased viscosity in the stomach and/or small intestine after ingestion. Accordingly, provided herein are methods for increasing the viscosity of stomach and/or small intestine contents, which include administering an ingestible composition to an animal. An animal can be any animal, as described above, and administration can be as described previously. Viscosity of stomach contents can be measured by any method known to those having ordinary skill in the art, including endoscopic techniques, imaging techniques (e.g., MRI), or in vivo or ex vivo viscosity measurements in e.g., control and treated animals.

Also provided are methods for promoting weight loss by administering an ingestible composition as provided herein to an animal. Administration can be as described previously. The amount and duration of such administration will depend on the individual's weight loss needs and health status, and can be evaluated by those having ordinary skill in the art. The animal's weight loss can be measured over time to determine if weight loss is occurring. Weight loss can be compared to a control animal not administered the ingestible composition.

Provided here are methods for improving weight reduction by at least 5% in an animal by orally administering to the animal a serving of a formed food having at least one soluble anionic fiber and a multivalent cation, wherein the weight reduction improvement is measured after four months of daily administration of the ingestible composition. The impovement in weight reduction ican be at least about 10%, at least about 15%, and at least about 20%.

The following examples are representative of the invention, and are not intended to be limiting to the scope of the invention.

### EXAMPLES

### Example 1

A cookie having a solid phase, e.g., a baked dough phase, containing a soluble anionic fiber blend and a fluid phase, e.g., jam phase containing a soluble calcium source deposited in the baked dough phase was produced.

The baked dough phase was prepared by adding BENEFAT® and lecithin to a premix of flour, cellulose, egg white, salt, leavening and flavors in a Hobart mixer and creaming by mixing at low speed for about 1 minute followed by high speed for about 2 minutes. The liquids were added to creamed mixture and blended at medium speed for about 2 minutes.

The fiber blend used contained about 46% sodium alginate LBA (ISP, San Diego, Calif.), about 39.6% sodium alginate GHB (ISP), and about 14.4% pectin (USP-L200, Kelco, San Diego, Calif.).

The fiber blend and glycerin were added to a separate bowl and combined. This combined fiber/glycerin material was added to the other ingredients in the Hobart mixer and was mixed on medium speed for about 1 minute. The resulting dough was then sheeted to desired thickness on a Rhondo sheeter and a dough pad measuring about 3 inched by about 6 inches was created.

The jam phase was prepared by adding a premixed BENEFAT® calcium source mixture to the jam base and mixed until uniformly mixed. A predetermined amount of the jam was then added onto the top surface of the cookie dough pad. The dough pad edges were wetted and sealed. Bars were baked at 325° F. for about 9 minutes, cut, cooled and the resulting cookies were individually packaged. The total caloric value of each cookie was about 50 kcal.

| | % Dough | % Total |
|---|---|---|
| Ingredient | Phase | Formulation |
| Flour all purpose | 29.140 | 12.165 |
| Cellulose, solka floc- | 6.980 | 2.914 |
| International Fiber Corp. | | |
| Powder egg white | 0.580 | 0.242 |
| Salt (NaCl) | 0.200 | 0.083 |
| Sodium Bicarbonate Grade #1 | 0.510 | 0.213 |
| Cookie Dough Flavor | 0.170 | 0.071 |
| BENEFAT | 2.060 | 0.860 |
| Lecithin | 0.640 | 0.267 |
| Polydextrose Litesse 70% syrup, | 15.870 | 6.625 |
| Ultra | | |
| Water | 11.830 | 4.939 |
| Liquid Vanilla flavor | 0.280 | 0.117 |
| sucralose, 25% liquid. | 0.090 | 0.038 |
| Potassium sorbate | 0.250 | 0.104 |
| Alginate fiber blend | 17.400 | 7.264 |
| Glycerine, Optim 99.7% USP | 14.000 | 5.845 |
| | 100.000 | 41.70 |

Fluid Phase:

| | % Fluid | % Total |
|---|---|---|
| Ingredient | Phase | Formulation |
| BENEFAT | 21.100 | 12.291 |
| Calcium Fumarate Trihydrate | 11.000 | 6.408 |
| Reduced Calorie Strawberry | 67.900 | 39.553 |
| Filling (Smuckers) | | |
| | 100.000 | 58.25 |

Control

Solid Phase:

| | % Dough | % Total |
|---|---|---|
| Ingredient | Phase | Formulation |
| Flour-all purpose | 29.140 | 12.530 |
| Cellulose, solka floc- | 6.980 | 3.001 |
| International Fiber Corp. | | |
| Powder egg white | 0.580 | 0.249 |
| Salt (NaCl) | 0.200 | 0.086 |
| Sodium Bicarbonate Grade #1 | 0.510 | 0.219 |
| Cookie Dough Flavor | 0.170 | 0.073 |
| BENEFAT | 19.450 | 8.364 |
| Lecithin | 0.640 | 0.275 |
| Polydextrose Litesse 70% syrup, | 15.870 | 6.824 |
| Ultra | | |
| Water | 11.830 | 5.087 |
| Liquid Vanilla flavor | 0.280 | 0.120 |
| sucralose, 25% liquid. | 0.090 | 0.039 |
| Potassium sorbate | 0.250 | 0.108 |
| Alginate fiber blend | 0.000 | 0.000 |
| Glycerine, Optim 99.7% USP | 14.000 | 6.020 |
| | 100.000 | 43.00 |

Jam Phase:

| | | % Total |
|---|---|---|
| Ingredient | % Fluid Phase | Formulations |
| BENEFAT | 32.100 | 19.260 |
| Reduced Calorie | 67.900 | 40.740 |
| Strawberry Filling | | |
| (SMUCKERS) | | |
| Total | 100.000 | 60.00 |

Intestinal Viscosity Measurement

Fully grown female Yucatan minipigs (Charles River Laboratories, Wilmington, MA), weighing about 90 kg, were fitted with indwelling silicone rubber sample ports (Omni Technologies, Inc., Greendale, IN) implanted in a surgically created dermal fistula at the ileocecal junction. The sample ports were sealed by a removable cap. These ports permit removal of samples of digesta as it passed from the ileum to the cecum. Additional details of this procedure are presented in B. Greenwood van-Meerveld et al., Comparison of Effects on Colonic Motility and Stool Characteristics Associated with Feeding Olestra and Wheat Bran to Ambulatory Mini-Pigs, Digestive Diseases and Sciences 44:1282-7 (1999), which is incorporated herein by reference.

Three Yucatan minipigs with the fistulas described above were housed in individual stainless steel pens in a windowless room maintained on a cycle of 12 hours of light and 12 hours of dark. They were conditioned to consume low fiber chow (Laboratory Mini-Pig Diet 5L80, PMI Nutritional International, Brentwood, Mo.). This chow contains about 5.3% fiber. The pigs were fed once each day, in the morning. Water was provided ad lib throughout the day.

Samples are taken from the ileal sample port immediately after feeding, and then at about 30 minute intervals for about 300 minutes. The volume of sample collected is about 50 to 130 ml. All samples are assayed for viscosity within 30 minutes after collection.

Samples of digesta were collected in sealed plastic containers. Viscosity of the digesta were measured with a Stevens QTS Texture Analyzer (Brookfield Engineering, Inc., Middleboro, Mass.). This instrument measures the relative viscosity of digesta by a back extrusion technique. The instrument included a stage plate, a 60 cm vertical tower, a mobile beam and a beam head that contained a load-cell. During back extrusion, the beam descended at a constant rate, and the force required to back extrude the sample was recorded over time. The sample containers were 5 cm deep spherical aluminum cups with an internal diameter of about 2.0 cm. The volume of the cup was about 20 ml. The spherical probe includes a 1.9 cm TEFLON brand ball mounted on a 2 mm threaded rod which is attached to the mobile beam. The diameters of the sample cup and probe allow for a wide range of viscosity (liquid to solid digesta) to be measured without approaching the maximum capacity of the rheometer (25 kg/peak force). During each test, the beam thrusts the probe into the test sample at a constant rate (12 cm/second) for a 2 cm stroke, forcing the sample to back-extrude around the equatorial region of the probe. The peak force for back extrusion at a controlled stroke rate is proportional to the viscosity of the sample. At each time point, 2-6 samples from each pig were tested, and the mean peak force WAS calculated and recorded.

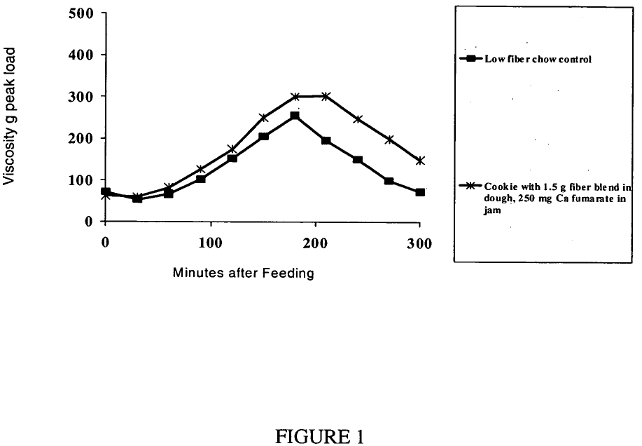

The test for effects of fiber containing cookies on viscosity was performed by providing each pig with its daily ration of low fiber chow (1400 g). Before feeding, one cookie was gently broken into four to six pieces and mixed into the chow. The animals have unlimited access to water during and after feeding. The effects of the cookie of this example containing fiber and calcium on intestinal viscosity is shown in FIG. 1. Each treatment was provided to each of three pigs on three separate days to yield nine replicates for each sample. Each point plotted in FIG. 1 is the mean of these nine determinations. The fiber and calcium containing cookie produced viscosities significantly greater than those produced by control chow (p<0.05, as measured by a two-tailed t-test) at the time points from 210 minutes through 300 minutes.

### Example 2

### Crispy Formulations

A variety of crispy formulations are prepared using the formulations as shown below followed by extrusion to make crispies:

To produce a batch of crispies, the ingredients are dry blended in a small ribbon blender. The resulting dry blend is transferred using a feeder, e.g., a K-Tron loss-in-weight feeder, into the hopper of an extruder, e.g., a Buhler Twin Screw Extruder configured with at least one heating unit, e.g., two Mokon barrel-heating units. Water is added as steam to the dry blend using a two barrel injection system for adding water and a second liquid into the barrel at variable rates. The blend is then mixed and cooked in the extruder. The hot pressurized product stream is forced through a die for expansion and then conveyed by vacuum to a fluid bed drier, e.g., Buhler fluid bed drier, and dried to the desired moisture content. The fluid bed drier can dry about 50 to about 100 kg/hour at temperatures from about 20- about 110° C.

Batch 1A-5367-54-01A

| | Ingredients | % |
|---|---|---|
| 1 | Rice Flour | 52.30 |
| 2 | Alginate LBA | 25.20 |
| 3 | Whey Protein Isolate | 20.00 |
| 4 | Starch | 2.00 |
| 5 | Salt | 0.50 |
| | Total | 100.00 |

Batch 2, 5367-54-02

| | Ingredients | % |
|---|---|---|
| 1 | Rice Flour | 54.30 |
| 2 | Alginate LBA | 25.20 |
| 3 | Whey Protein Isolate | 12.00 |
| 4 | Starch | 8.00 |
| 5 | Salt | 0.50 |
| | Total | 100.00 |

Batch 3, 5367-54-03

| | Ingredients | % |
|---|---|---|
| 1 | Rice Flour | 66.30 |
| 2 | Alginate LBA | 25.20 |
| 3 | Whey Protein Isolate | 00.00 |
| 4 | Starch | 8.00 |
| 5 | Salt | 0.50 |
| | Total | 100.00 |

Batch 4, 5367-54-04

| | Ingredients | % |
|---|---|---|
| 1 | Rice Flour | 54.30 |
| 2 | Alginate LBA | 12.00 |
| 3 | Alginate DPB | 6.00 |
| 4 | Alginate KTHV | 7.20 |
| 5 | Whey Protein Isolate | 12.00 |
| 6 | Starch | 8.00 |
| 7 | Salt | 0.50 |
| | Total | 100.00 |

Batch #1, repeat 5367-54-04

| | Ingredients | % |
|---|---|---|
| 1 | Rice Flour | 54.30 |
| 2 | Alginate LBA | 12.00 |
| 3 | Alginate DPB | 6.00 |
| 4 | Alginate KTHV | 7.20 |
| 5 | Whey Protein Isolate BiPro | 12.00 |
| 6 | Wheat Starch | 8.00 |
| 7 | Salt | 0.50 |
| | Total | 100.00 |

Batch #2, based on 5367-54-04

| Formula # 5981-04-01 | | |
|---|---|---|
| | Ingredients | % |
| 1 | Rice Flour | 54.30 |
| 2 | Alginate DPB | 25.20 |
| 3 | Whey Protein Isolate BiPro | 12.00 |
| 4 | Whey Starch | 8.00 |
| 5 | Salt | 0.50 |
| | Total | 100.00 |

Batch #3, Same as Batch #2 (5981-04-01), replacing DPB with KTHV
Formula # 5981-04-02

| | Ingredients | % |
|---|---|---|
| 1 | Rice Flour | 54.30 |
| 2 | Alginate KTHV | 25.20 |
| 3 | Whey Protein Isolate BiPro | 12.00 |
| 4 | Whey Starch | 8.00 |
| 5 | Salt | 0.50 |
| | Total | 100.00 |

Batch #4: Based on 5981-04-01, increased alginate by 25%
Formula # 5981-04-03

| | Ingredients | % |
|---|---|---|
| 1 | Rice Flour | 56.00 |
| 2 | Alginate KTHV | 31.50 |
| 3 | Whey Protein Isolate BiPro | 8.00 |
| 4 | Corn Starch | 4.00 |
| 5 | Salt | 0.50 |
| | Total | 100.00 |

Batch #5: Based on 5981-04-01, increased alginate by 50%
Formula # 5981-04-04

| | Ingredients | % |
|---|---|---|
| 1 | Rice Flour | 49.70 |
| 2 | Alginate KTHV | 37.80 |
| 3 | Whey Protein Isolate BiPro | 8.00 |
| 4 | Corn Starch | 4.00 |
| 5 | Salt | 0.50 |
| | Total | 100.00 |

Formula # 5981-04-05

| | Ingredients | % |
|---|---|---|
| 1 | Rice Flour | 46.00 |
| 2 | Alginate KTHV | 31.50 |
| 3 | Whey Protein Isolate BiPro | 8.00 |
| 4 | Corn Starch | 4.00 |
| 5 | Salt | 0.50 |
| 6 | Inulin | 10.00 |
| | Total | 100.00 |

Formula # 5981-04-06

| | Ingredients | % |
|---|---|---|
| 1 | Corn Meal | 56.00 |
| 2 | Alginate KTHV | 31.50 |
| 3 | Whey Protein Isolate BiPro | 8.00 |
| 4 | Corn Starch | 4.00 |
| 5 | Salt | 0.50 |
| | Total | 100.00 |

Formula # 5981-04-07

| | Ingredients | % |
|---|---|---|
| 1 | Corn Cone | 64.00 |
| 2 | Alginate KTHV | 31.50 |
| 3 | Whey Protein Isolate BiPro | 8.00 |
| 4 | Corn Starch | 0.00 |
| 5 | Salt | 0.50 |
| 6 | Inulin F97 | 0.00 |
| | Total | 100.00 |

Formula # 5981-04-08

| | Ingredients | % |
|---|---|---|
| 1 | Corn Cone | 53.70 |
| 2 | Alginate KTHV | 37.80 |
| 3 | Whey Protein Isolate BiPro | 0.00 |
| 4 | Corn Starch | 0.00 |
| 5 | Salt | 0.50 |
| 6 | Inulin F97 | 8.00 |
| | Total | 100.00 |

Batch #10
Formula # 5981-04-09

| | Ingredients | % |
|---|---|---|
| 1 | Corn Cone | 46.70 |
| 2 | Alginate KTHV | 42.80 |
| 3 | Whey Protein Isolate BiPro | 0.00 |
| 4 | Corn Starch | 0.00 |
| 5 | Salt | 0.50 |
| 6 | Inulin F97 | 10.00 |
| | Total | 100.00 |

Batch #11 Formula # 5981-04-10

| | Ingredients | % |
|---|---|---|
| 1 | Com Cone | 49.50 |
| 2 | Alginate KTHV | 50.00 |
| 3 | Whey Protein Isolate BiPro | 0.00 |
| 4 | Corn Starch | 0.00 |
| 5 | Salt | 0.50 |
| 6 | Inulin F97 | 0.00 |
| | Total | 100.00 |

Batch #12, same formula as 5981-04-07, used different die to make curls
Formula # 5981-04-11

| | Ingredients | % |
|---|---|---|
| 1 | Corn Cone | 56.00 |
| 2 | Alginate KTHV | 31.50 |
| 3 | Whey Protein Isolate BiPro | 4.00 |
| 4 | Corn Starch | 0.00 |
| 5 | Salt | 0.50 |
| 6 | Inulin F97 | 8.00 |
| | Total | 100.00 |

Batch #13 = batch #10 5981-04-09
Formula # 5981-04-12

| | Ingredients | % |
|---|---|---|
| 1 | Corn Cone | 46.70 |
| 2 | Alginate KTHV | 42.80 |
| 3 | Whey Protein Isolate BiPro | 0.00 |
| 4 | Corn Starch | 0.00 |
| 5 | Salt | 0.50 |
| 6 | Inulin F97 | 10.00 |
| | Total | 100.00 |

Batch #14: same as batch #4,5981-04-03 except replaced KTHV with DPB
Formula # 5981-15-01

| | Ingredients | % |
|---|---|---|
| 1 | Rice Flour | 56.00 |
| 2 | Alginate DPB | 31.50 |
| 3 | Whey Protein Isolate BiPro | 8.00 |
| 4 | Corn Starch | 4.00 |
| 5 | Salt | 0.50 |
| | Total | 100.00 |

Batch #15: based on batch #14
Formula # 5981-15-02

| | Ingredients | % |
|---|---|---|
| 1 | Rice Flour | 56.00 |
| 2 | Alginate DPB | 31.50 |
| 3 | Whey Protein Isolate BiPro | 4.00 |
| 4 | Corn Starch | 3.00 |
| 5 | Salt | 0.50 |
| 6 | Cocoa | 5.00 |
| | Total | 100.00 |

Batch #16: based on batch #14
Formula # 5981-15-03

| | Ingredients | % |
|---|---|---|
| 1 | Rice Flour | 56.00 |
| 2 | Alginate DPB | 31.50 |
| 3 | Whey Protein Isolate BiPro | 4.00 |
| 4 | Corn Starch | 4.00 |
| 5 | Salt | 0.50 |
| 6 | Micro-crystalline Cellolose | 4.00 |
| | Total | 100.00 |

Batch #17: based on batch 14
Formula # 5981-15-04

| | Ingredients | % |
|---|---|---|
| 1 | Rice Flour | 56.00 |
| 2 | Alginate DPB | 31.50 |
| 3 | Whey Protein Isolate BiPro | 7.00 |
| 4 | Corn Starch | 4.00 |
| 5 | Salt | 0.50 |
| 6 | Glycerine | 1.00 |
| | Total | 100.00 |

Batch #18: based on batch # 14
Formula # 5981-15-05

| | Ingredients | % |
|---|---|---|
| 1 | Rice Flour | 56.00 |
| 2 | Alginate DPB | 31.50 |
| 3 | Whey Protein Isolate BiPro | 4.00 |
| 4 | Corn Starch | 3.00 |
| 5 | Salt | 0.50 |
| 6 | Oil | 5.00 |
| | Total | 100.00 |

Batch #19: based on batch # 14
Formula # 5981-15-06

| | Ingredients | % |
|---|---|---|
| 1 | Rice Flour | 56.00 |
| 2 | Alginate DPB | 31.50 |
| 3 | Whey Protein Isolate BiPro | 7.95 |
| 4 | Corn Starch | 4.00 |
| 5 | Salt | 0.50 |
| 6 | Sucrolose | 0.05 |
| | Total | 100.00 |

Batch #20: based on batch # 14
Formula # 5981-15-07

| | Ingredients | % |
|---|---|---|
| 1 | Rice Flour | 56.00 |
| 2 | Alginate DPB | 31.50 |
| 3 | Whey Protein Isolate BiPro | 4.00 |
| 4 | Corn Starch | 3.00 |
| 5 | Salt | 0.50 |
| 6 | Trehalose | 5.00 |
| | Total | 100.00 |

Batch #21: based on batch # 7, 5981-04-06
Formula # 5981-15-08

| | Ingredients | % |
|---|---|---|
| 1 | Corn Cone | 56.00 |
| 2 | Alginate DPB | 31.50 |
| 3 | Whey Protein Isolate BiPro | 8.00 |
| 4 | Corn Starch | 4.00 |
| 5 | Salt | 0.50 |
| | Total | 100.00 |

Batch #22: same as batch # 21
Formula # 5981-15-09

| | Ingredients | % |
|---|---|---|
| 1 | Corn Cone 2 | 56.00 |
| 2 | Alginate DPB | 31.50 |
| 3 | Whey Protein Isolate BiPro | 8.00 |
| 4 | Corn Starch | 4.00 |
| 5 | Salt | 0.50 |
| | Total | 100.00 |

Batch #25
Formula # 5981-15-12

| | Ingredients | % |
|---|---|---|
| 1 | Corn Cone | 54.00 |
| 2 | Alginate DPB | 31.50 |
| 3 | Whey Protein Isolate BiPro | 2.00 |
| 4 | Corn Starch | 2.00 |
| 5 | Salt | 0.50 |
| 6 | Corn Fiber | 10.00 |
| | Total | 100.00 |

Formula # 5981-15-15

| | Ingredients | % |
|---|---|---|
| 1 | Rice Flour | 56.50 |
| 2 | Alginate DPB | 31.50 |
| 3 | Whey Protein Isolate BiPro | 4.00 |
| 4 | Corn Starch | 3.00 |
| 5 | Fractionated Canola Oil | 5.00 |
| | Total | 100.00 |

Formula # 5981-15-25

| | Ingredients | % |
|---|---|---|
| 1 | Rice Flour | 88.00 |
| 2 | Whey Protein Isolate BiPro | 4.00 |
| 3 | Corn Starch | 3.00 |
| 4 | Fractionated Canola Oil | 5.00 |
| | Total | 100.00 |

### Example 3

### Bars

Nutritional bars with a nougat center were prepared by the following procedure. All liquid ingredients were placed in a mixer bowl with the paddle attachment. After one mixing for one minute, the dry ingredients were added except proteins and mixing was continued to mix on low speed. After 1 minute, proteins were added to the dough, and mixing was continued on low to medium speed for an additional 2 minutes. The dough was then formed into desired shapes and sizes either manually or through an extruder. Bars were coated with coatings of desired flavors and/or colors by submersion into melted (120° F.) compound coating, or into chocolate that has been melted (120° F.) and tempered (90° F.). Coated bars were allowed to cool to harden the coating, and were then packaged.

Chocolate Peanut Butter
Serving size: 50 g

| # | Ingredient | Percentage |
|---|---|---|
| 1 | Chocolate Coating | 12.50 |
| 2 | HFCS | 20.00 |
| 3 | Glycerine | 12.50 |
| 4 | Water | 10.00 |
| 5 | Canola Oil | 5.00 |
| 6 | Inulin | 5.00 |
| 7 | Tricalcium Phosphate | 2.50 |
| 8 | Calcium Caseinate | 10.00 |
| 9 | Whey Protein Isolate | 12.50 |
| 10 | Psyllium | 10.00 |
| | Total | 100.00 |

Chocolate Peanut Butter
Formula # 5367-45-33
Serving size 55 g, 5 g alginate

| # | Ingredient | Percentage |
|---|---|---|
| 1 | Sugar-Free Choc Coating | 18.20 |
| 2 | High fructose corn syrup | 13.00 |
| 3 | Maltitol | 9.90 |
| 4 | Glycerine | 5.00 |
| 5 | Peanut Butter | 3.00 |
| 6 | Canola Oil | 3.00 |
| 7 | Peanut Butter Flavor | 3.00 |
| 8 | Vanilla | 0.50 |
| | Mix for 0.5 min | |
| 9 | Alginate | 9.10 |
| | Mix for 1 min | |
| 10 | Erythritol | 8.00 |
| 11 | Inulin | 3.00 |
| 12 | Peanut Flour | 5.00 |
| | Mix for 0.5 min | |
| 13 | Tricalcium Phosphate | 0.00 |
| 14 | Calcium Carbonate | 0.00 |
| 15 | Whey Protein Isolate | 8.30 |
| 16 | Peanuts | 11.00 |
| | Mix for 1 min | |
| | Total | 100.00 |

Chocolate Peanut Butter
Formula # 5367-45-32
Serving size 55 g, 5 g alginate

| # | Ingredient | Percentage |
|---|---|---|
| 1 | Sugar-Free Choc Coating | 18.20 |
| 2 | High fructose corn syrup | 12.00 |
| 3 | Maltitol | 9.90 |
| 4 | Glycerine | 3.00 |
| 5 | Peanut Butter | 3.00 |
| 6 | Peanut Butter Flavor | 3.00 |
| 7 | Vanilla | 0.50 |
| | Mix for 0.5 min | |
| 8 | Alginate | 9.10 |
| | Mix for 1 min | |
| 9 | Erythritol | 8.00 |
| 10 | Inulin | 5.00 |
| 11 | Peanut Flour | 5.00 |
| 12 | Hydrolyzed Whey Isolate | 4.00 |
| 13 | Sucrolose | 0.00 |
| | Mix for 0.5 min | |
| 14 | Tricalcium Phosphate | 0.00 |
| 15 | Calcium Carbonate | 0.00 |
| 16 | Whey Protein Isolate | 8.30 |
| 17 | Peanuts | 11.00 |
| | Mix for 1 min | |
| | Total | 100.00 |

Aw = 0.521

Chocolate Peanut Butter
Formula # 5367-45-01
Serving size 45 g, contains 200 mg Ca, 5% alginate/pectin, 3 g inulin

| # | Ingredient | Percentage |
|---|---|---|
| 1 | Chocolate Coating | 15.00 |
| 2 | High fructose corn syrup | 9.50 |
| 3 | Water | 9.50 |
| 4 | Glycerine | 7.00 |
| 5 | Peanut Flour | 8.00 |
| 6 | Peanut Butter | 10.00 |
| 7 | Peanut Flavor | 1.15 |
| 8 | Inulin | 6.70 |
| 9 | Alginate | 2.50 |
| 10 | Pectin | 2.50 |
| 11 | Soy Protein Isolate | 8.00 |
| 12 | Calcium Caseinate | 7.00 |
| 13 | Whey Protein Isolate | 7.00 |
| 14 | Peanuts | 6.15 |
| 15 | Tricalcium Phosphate | 1.15 |
| | Total | 100.00 |

Chocolate Raspberry
Formula # 5367-44-03
Serving size 40 g, contains 200 mg Ca, 1 g alginate, 1 g pectin, 3 g inulin

| # | Ingredient | Percentage |
|---|---|---|
| 1 | Sugar-Free Choc Coating | 18.00 |
| 2 | Water | 12.60 |
| 3 | Glycerine | 8.00 |
| 4 | Canola Oil | 7.00 |
| 5 | Raspberry Flavor | 1.50 |
| 6 | Raspberry Flavor | 0.75 |
| 7 | White Chocolate Flavor | 0.35 |
| 8 | Vanilla Flavor | 1.00 |
| 9 | Sucrolose | 0.20 |
| 10 | Red Color | 0.10 |
| 11 | Inulin | 5.45 |
| 12 | Erythritol | 8.00 |
| 13 | Fructose | 4.00 |
| 14 | Malic Acid | 0.30 |
| 15 | Alginate | 2.15 |
| 16 | Pectin | 3.04 |
| 17 | Soy Protein Isolate | 5.50 |
| 18 | Calcium Caseinate | 5.50 |
| 19 | Whey Protein Isolate | 8.00 |
| 20 | Tricalcium Phosphate | 0.96 |
| 21 | Dried Raspberry | 3.00 |
| 22 | Soy Crisps, 80% protein | 4.60 |
| | Total | 100.00 |

Chocolate Peanut Butter
Formula # 5367-45-13
Serving size 55 g, no calcium, 5 g alginate

| # | Ingredient | Percentage |
|---|---|---|
| 1 | Sugar-Free Choc Coating | 20.00 |
| 2 | Corn Syrup | 14.50 |
| 3 | Glycerine | 12.00 |
| 4 | Peanut Butter | 10.00 |
| 5 | Peanut Butter Flavor | 2.00 |
| 6 | Maltodextrin | 0.00 |
| 7 | Erythritol | 6.00 |
| 8 | Alginate | 10.70 |
| 9 | Pectin | 0.00 |
| 10 | Peanut Flour | 3.40 |
| 11 | Tricalcium Phosphate | 0.00 |
| 12 | Soy Protein Isolate | 4.00 |
| 13 | Calcium Caseinate | 4.00 |
| 14 | Whey Protein Isolate | 4.00 |
| 16 | Peanuts | 9.40 |
| 17 | Soy Crisps | 0.00 |
| | Total | 100.00 |

Chocolate Peanut Butter
Formula # 5367-45-14
Serving size 55 g, no calcium, 5 g alginate
LBA alginate

| # | Ingredient | Percentage |
|---|---|---|
| 1 | Sugar-Free Choc Coating | 20.00 |
| 2 | Corn Syrup | 14.50 |
| 3 | Glycerine | 12.00 |
| 4 | Peanut Butter | 10.00 |
| 5 | Peanut Butter Flavor | 2.00 |
| 6 | Maltodextrin | 0.00 |
| 7 | Erythritol | 6.00 |
| 8 | Alginate | 10.70 |
| 9 | Pectin | 0.00 |
| 10 | Peanut Flour | 3.40 |
| 11 | Tricalcium Phosphate | 0.00 |
| 12 | Soy Protein Isolate | 4.00 |
| 13 | Calcium Caseinate | 4.00 |
| 14 | Whey Protein Isolate | 4.00 |
| 16 | Peanuts | 9.40 |
| 17 | Soy Crisps | 0.00 |
| | Total | 100.00 |

Aw = 0.383

Chocolate Peanut Butter
Formula # 5367-45-15
Serving size 55 g, no calcium, 5 g alginate
GHB alginate

| # | Ingredient | Percentage |
|---|---|---|
| 1 | Sugar-Free Choc Coating | 20.00 |
| 2 | Corn Syrup | 14.50 |
| 3 | Glycerine | 8.00 |
| 4 | Peanut Butter | 10.00 |
| 5 | Peanut Butter Flavor | 2.00 |
| 6 | Maltodextrin | 0.00 |
| 7 | Erythritol | 6.00 |
| 8 | Alginate | 10.70 |
| 9 | Pectin | 0.00 |
| 10 | Peanut Flour | 3.40 |
| 11 | Tricalcium Phosphate | 0.00 |
| 12 | Soy Protein Isolate | 5.00 |
| 13 | Calcium Caseinate | 5.00 |
| 14 | Whey Protein Isolate | 5.00 |
| 15 | Peanuts | 10.40 |
| 16 | Soy Crisps | 0.00 |
| | Total | 100.00 |

Aw = 0.383

Chocolate Peanut Butter
Formula # 5367-45-16
Serving size 55 g, no calcium, 5 g alginate (LBA alginate)

| # | Ingredient | Percentage |
|---|---|---|
| 1 | Sugar-Free Choc Coating | 20.00 |
| 2 | Corn Syrup | 14.50 |
| 3 | Glycerine | 8.00 |
| 4 | Peanut Butter | 10.00 |
| 5 | Peanut Butter Flavor | 2.00 |
| 6 | Maltodextrin | 0.00 |
| 7 | Erythritol | 6.00 |
| 8 | Alginate | 10.70 |
| 9 | Pectin | 0.00 |
| 10 | Peanut Flour | 3.40 |
| 11 | Tricalcium Phosphate | 0.00 |
| 12 | Soy Protein Isolate | 5.00 |
| 13 | Calcium Caseinate | 5.00 |
| 14 | Whey Protein Isolate | 5.00 |
| 15 | Peanuts | 10.40 |
| 16 | Soy Crisps | 0.00 |
| | Total | 100.00 |

Aw = 0.383

Chocolate Peanut Butter
Formula # 5367-45-17
Serving size 55 g, no calcium
Spray Dry Batch # 1

| # | Ingredient | Percentage |
|---|---|---|
| 1 | Sugar-Free Choc Coating | 20.00 |
| 2 | Corn Syrup | 17.00 |
| 3 | Glycerine | 3.00 |
| 4 | Peanut Butter | 10.00 |
| 5 | Peanut Butter Flavor | 2.00 |
| 6 | Maltodextrin | 0.00 |
| 7 | Erythritol | 4.00 |
| 8 | Alginate | 21.81 |
| 9 | Pectin | 0.00 |
| 10 | Peanut Flour | 3.40 |
| 11 | Tricalcium Phosphate | 0.00 |
| 12 | Soy Protein Isolate | 3.50 |
| 13 | Calcium Caseinate | 3.50 |
| 14 | Whey Protein Isolate | 3.50 |
| 15 | Peanuts | 8.29 |
| 16 | Soy Crisps | 0.00 |
| | Total | 100.00 |

Aw 0.519

Chocolate Peanut Butter
Formula # 5367-45-18
Serving size 55 g, no calcium, 5 g alginate
LBA

| # | Ingredient | Percentage |
|---|---|---|
| 1 | Sugar-Free Choc Coating | 20.00 |
| 2 | Corn Syrup | 14.00 |
| 3 | Glycerine | 8.00 |
| 4 | Peanut Butter | 10.00 |
| 5 | Peanut Butter Flavor | 2.00 |
| 6 | Maltodextrin | 10.00 |
| 7 | Erythritol | 4.00 |
| 8 | Alginate | 10.70 |
| 9 | Pectin | 0.00 |
| 10 | Peanut Flour | 3.40 |
| 11 | Tricalcium Phosphate | 0.00 |
| 12 | Soy Protein Isolate | 3.00 |
| 13 | Calcium Caseinate | 3.00 |
| 14 | Whey Protein Isolate | 3.00 |
| 15 | Peanuts | 7.90 |
| 16 | Soy Crisps | 0.00 |
| | Total | 99.00 |

Aw = 0.340

Chocolate Peanut Butter
Formula # 5367-45-19
Serving size 55 g, no calcium
Spray Dry Batch # 2

| # | Ingredient | Percentage |
|---|---|---|
| 1 | Sugar-Free Choc Coating | 20.00 |
| 2 | Corn Syrup | 14.00 |
| 3 | Glycerine | 8.00 |
| 4 | Peanut Butter | 10.00 |
| 5 | Peanut Butter Flavor | 2.00 |
| 6 | Maltodextrin | 10.00 |
| 7 | Erythritol | 4.00 |
| 8 | Alginate | 10.70 |
| 9 | Pectin | 0.00 |
| 10 | Peanut Flour | 3.40 |
| 11 | Tricalcium Phosphate | 0.00 |
| 12 | Soy Protein Isolate | 3.00 |
| 13 | Calcium Caseinate | 3.00 |
| 14 | Whey Protein Isolate | 3.00 |
| 15 | Peanuts | 7.90 |
| 16 | Soy Crisps | 0.00 |
| | Total | 99.00 |

Aw = 0.340

Chocolate Peanut Butter
Formula # 5367-45-20
Serving size 55 g, no calcium
Spray Dry Batch # 3

| # | Ingredient | Percentage |
|---|---|---|
| 1 | Sugar-Free Choc Coating | 20.00 |
| 2 | Corn Syrup | 20.00 |
| 3 | Glycerine | 0.00 |
| 4 | Maltitol | 16.70 |
| 5 | Peanut Butter | 10.00 |
| 6 | Peanut Butter Flavor | 2.00 |
| 7 | Maltodextrin | 0.00 |
| 8 | Erythritol | 0.00 |
| 9 | Alginate | 16.00 |
| 10 | Pectin | 0.00 |
| 11 | Peanut Flour | 2.30 |
| 12 | Tricalcium Phosphate | 0.00 |
| 13 | Soy Protein Isolate | 0.00 |
| 14 | Calcium Caseinate | 0.00 |
| 15 | Whey Protein Isolate | 0.00 |
| 16 | Peanuts | 8.00 |
| 17 | Soy Crisps | 0.00 |
| | Total | 95.00 |

Chocolate Peanut Butter
Formula # 5367-45-21
Serving size 55 g, no calcium
Spray Dry Batch # 4

| # | Ingredient | Percentage |
|---|---|---|
| 1 | Sugar-Free Choc Coating | 20.00 |
| 2 | Corn Syrup | 20.00 |
| 3 | Glycerine | 0.00 |
| | Maltitol | 16.10 |
| 4 | Peanut Butter | 10.00 |
| 5 | Peanut Butter Flavor | 2.00 |
| 6 | Maltodextrin | 0.00 |
| 7 | Erythritol | 0.00 |
| 8 | Alginate | 21.40 |
| 9 | Pectin | 0.00 |
| 10 | Peanut Flour | 2.30 |
| 11 | Tricalcium Phosphate | 0.00 |
| 12 | Soy Protein Isolate | 0.00 |
| 13 | Calcium Caseinate | 0.00 |
| 14 | Whey Protein Isolate | 0.00 |
| 15 | Peanuts | 3.20 |
| 16 | Soy Crisps | 0.00 |
| | Total | 95.00 |

Chocolate Peanut Butter
Formula # 5367-45-22
Serving size 55 g, no calcium
Spray Dry Batch # 5

| # | Ingredient | Percentage |
|---|---|---|
| 1 | Sugar-Free Choc Coating | 20.00 |
| 2 | Corn Syrup | 20.00 |
| 3 | Glycerine | 0.00 |
| 4 | Maltitol | 16.70 |
| 5 | Peanut Butter | 10.00 |
| 6 | Peanut Butter Flavor | 2.00 |
| 7 | Maltodextrin | 0.00 |
| 8 | Erythritol | 0.00 |
| 9 | Alginate | 16.00 |
| 10 | Pectin | 0.00 |
| 11 | Peanut Flour | 2.30 |
| 12 | Tricalcium Phosphate | 0.00 |
| 13 | Soy Protein Isolate | 0.00 |
| 14 | Calcium Caseinate | 0.00 |
| 15 | Whey Protein Isolate | 0.00 |
| 16 | Peanuts | 8.00 |
| 17 | Soy Crisps | 0.00 |
| | Total | 95.00 |

Chocolate Peanut Butter
Formula # 5367-45-23
Serving size 55 g, no calcium
Spray Dry Batch # 6
17% coating

| # | Ingredient | Percentage |
|---|---|---|
| 1 | Sugar-Free Choc Coating | 20.00 |
| 2 | Corn Syrup | 20.00 |
| 3 | Glycerine | 0.00 |
| | Maltitol | 16.70 |
| 4 | Peanut Butter | 10.00 |
| 5 | Peanut Butter Flavor | 2.00 |
| 6 | Maltodextrin | 0.00 |
| 7 | Erythritol | 0.00 |
| 8 | Alginate | 16.00 |
| 9 | Pectin | 0.00 |
| 10 | Peanut Flour | 2.30 |
| 11 | Tricalcium Phosphate | 0.00 |
| 12 | Soy Protein Isolate | 0.00 |
| 13 | Calcium Caseinate | 0.00 |
| 14 | Whey Protein Isolate | 0.00 |
| 15 | Peanuts | 8.00 |
| 16 | Soy Crisps | 0.00 |
| | Total | 95.00 |

Chocolate Peanut Butter
Formula # 5367-45-24
Serving size 55 g, no calcium
Spray Dry Batch # 1

| # | Ingredient | Percentage |
|---|---|---|
| 1 | Sugar-Free Choc Coating | 20.00 |
| 2 | Corn Syrup | 17.00 |
| 3 | Glycerine | 0.00 |
| 4 | Maltitol | 16.70 |
| 5 | Peanut Butter | 10.00 |
| 6 | Peanut Butter Flavor | 2.00 |
| 7 | Maltodextrin | 0.00 |
| 8 | Erythritol | 0.00 |
| 9 | Alginate | 21.81 |
| 10 | Pectin | 0.00 |
| 11 | Peanut Flour | 3.40 |
| 12 | Tricalcium Phosphate | 0.00 |
| 13 | Soy Protein Isolate | 1.00 |
| 14 | Calcium Caseinate | 1.00 |
| 15 | Whey Protein Isolate | 1.00 |
| 16 | Peanuts | 6.09 |
| 17 | Soy Crisps | 0.00 |
| | Total | 100.00 |

Chocolate Peanut Butter
Formula # 5367-45-25
Serving size 55 g, 5 g alginate
LBA (alginate)

| # | Ingredient | Percentage |
|---|---|---|
| 1 | Sugar-Free Choc Coating | 20.00 |
| 2 | Corn Syrup | 14.00 |
| 3 | Glycerine | 8.00 |
| 4 | Peanut Butter | 10.00 |
| 5 | Peanut Butter Flavor | 2.00 |
| 6 | Maltodextrin | 5.00 |
| | Inulin | 5.00 |
| 7 | Erythritol | 4.00 |
| 8 | Alginate | 10.70 |
| 9 | Pectin | 0.00 |
| 10 | Peanut Flour | 3.40 |
| 11 | Tricalcium Phosphate | 0.47 |
| | Calcium Carbonate | 0.46 |
| 12 | Soy Protein Isolate | 3.00 |
| 13 | Calcium Caseinate | 3.00 |
| 14 | Whey Protein Isolate | 3.00 |
| 15 | Peanuts | 7.98 |
| 16 | Soy Crisps | 0.00 |
| | Total | 100.00 |

Chocolate Peanut Butter
Formula # 5367-45-26
Serving size 55 g, 5 g alginate

| # | Ingredient | Percentage |
|---|---|---|
| 1 | Sugar-Free Choc Coating | 20.00 |
| 2 | Corn Syrup | 16.00 |
| 3 | Glycerine | 6.00 |
| 4 | Peanut Butter | 10.00 |
| 5 | Peanut Butter Flavor | 2.00 |
| 6 | Maltodextrin | 5.00 |
| | Inulin | 5.00 |
| 7 | Erythritol | 6.00 |
| 8 | Alginate | 10.70 |
| 9 | Pectin | 0.00 |
| 10 | Peanut Flour | 3.40 |
| 11 | Tricalcium Phosphate | 0.47 |
| | Calcium Carbonate | 0.46 |
| 12 | Soy Protein Isolate | 3.00 |
| 13 | Calcium Caseinate | 3.00 |
| 14 | Whey Protein Isolate | 3.00 |
| 15 | Peanuts | 7.98 |
| 16 | Soy Crisps | 0.00 |
| | Total | 102.00 |

Chocolate Peanut Butter, Jan. 26, 2004
Formula # 5367-45-27
Serving size 55 g, 5 g alginate

| # | Ingredient | Percentage |
|---|---|---|
| 1 | Sugar-Free Choc Coating | 20.00 |
| 2 | Corn Syrup | 15.00 |
| 3 | Glycerine | 6.00 |
| 4 | Peanut Butter | 10.00 |
| 5 | Peanut Butter Flavor | 2.00 |
| 6 | Maltodextrin | 5.00 |
| | Inulin | 5.00 |
| 7 | Erythritol | 5.00 |
| 8 | Alginate | 10.70 |
| 9 | Pectin | 0.00 |
| 10 | Peanut Flour | 3.40 |
| 11 | Tricalcium Phosphate | 0.47 |
| | Calcium Carbonate | 0.46 |
| 12 | Soy Protein Isolate | 3.00 |
| 13 | Calcium Caseinate | 3.00 |
| 14 | Whey Protein Isolate | 3.00 |
| 15 | Peanuts | 7.98 |
| 16 | Soy Crisps | 0.00 |
| | Total | 100.00 |

Chocolate Peanut Butter
Formula # 5367-45-30B, LBA
Serving size 55 g, 5 g alginate

| # | Ingredient | Percentage |
|---|---|---|
| 1 | Sugar-Free Choc Coating | 18.20 |
| 2 | HFCS | 10.00 |
| 3 | Glycerine | 7.00 |
| 4 | Maltitol | 7.00 |
| 5 | Peanut Butter | 3.00 |
| 6 | Peanut Butter Flavor | 3.00 |
| 7 | Vanilla | 0.50 |
| 8 | Sucrolose | 0.01 |
| 9 | Inulin | 5.00 |
| 10 | Erythritol | 8.00 |
| 11 | Alginate | 9.10 |
| 12 | Peanut Flour | 5.00 |
| 13 | Hydrolyzed Whey Isolate | 7.00 |
| 14 | Tricalcium Phosphate | 0.00 |
| 15 | Calcium Carbonate | 0.00 |
| 16 | Whey Protein Isolate | 8.50 |
| 17 | Peanuts | 8.69 |
| | Total | 100.00 |

Aw = 0.402

Chocolate Peanut Butter
Formula # 5367-45-28
Serving size 55 g, 5 g alginate

| # | Ingredient | Percentage |
|---|---|---|
| 1 | Sugar-Free Choc Coating | 20.00 |
| 2 | Corn Syrup | 20.00 |
| 3 | Glycerine | 6.00 |
| 4 | Maltitol | 5.00 |
| 5 | Peanut Butter | 8.00 |
| 6 | Peanut Butter Flavor | 2.00 |
| 7 | Maltodextrin | 0.00 |
| 8 | Inulin | 0.00 |
| 9 | Erythritol | 5.00 |
| 10 | Alginate | 0.00 |
| 11 | Pectin | 0.00 |
| 12 | Spray Dry Alginate | 14.26 |
| 13 | Peanut Flour | 3.40 |
| 14 | Tricalcium Phosphate | 0.00 |
| 15 | Calcium Carbonate | 0.00 |
| 16 | Soy Protein Isolate | 3.00 |
| 17 | Calcium Caseinate | 3.00 |
| 18 | Whey Protein Isolate | 3.00 |
| 19 | Peanuts | 7.34 |
| 20 | Soy Crisps | 0.00 |
| | Total | 100.00 |

Chocolate Peanut Butter
Formula # 5367-45-29
Serving size 55 g, 5 g alginate

| # | Ingredient | Percentage |
|---|---|---|
| 1 | Sugar-Free Choc Coating | 20.00 |
| 2 | Corn Syrup | 20.00 |
| 3 | Glycerine | 6.00 |
| 4 | Maltitol | 5.00 |
| 5 | Peanut Butter | 8.00 |
| 6 | Peanut Butter Flavor | 2.00 |
| 7 | Maltodextrin | 4.76 |
| 8 | Inulin | 0.00 |
| 9 | Erythritol | 5.00 |
| 10 | Alginate | 0.00 |
| 11 | Pectin | 0.00 |
| 12 | Spray Dry Alginate | 9.50 |
| 13 | Peanut Flour | 3.40 |
| 14 | Tricalcium Phosphate | 0.00 |
| 15 | Calcium Carbonate | 0.00 |
| 16 | Soy Protein Isolate | 3.00 |
| 17 | Calcium Caseinate | 3.00 |
| 18 | Whey Protein Isolate | 3.00 |
| 19 | Peanuts | 7.34 |
| 20 | Soy Crisps | 0.00 |
| | Total | 100.00 |

Chocolate Peanut Butter
Formula # 5367-45-34
Serving size 55 g, 5 g alginate

| # | Ingredient | Percentage |
|---|---|---|
| 1 | Sugar-Free Choc Coating | 20.00 |
| 2 | Corn Syrup | 15.00 |
| 3 | Glycerine | 6.00 |
| 4 | Peanut Butter | 10.00 |
| 5 | Peanut Butter Flavor | 2.00 |
| 6 | Maltodextrin | 5.00 |
| | Inulin | 5.00 |
| 7 | Erythritol | 5.00 |
| 8 | Alginate | 10.70 |
| 9 | Pectin | 0.00 |
| 10 | Peanut Flour | 3.40 |
| 11 | Tricalcium Phosphate | 0.47 |
| | Calcium Carbonate | 0.46 |
| 12 | Soy Protein Isolate | 3.00 |
| 13 | Calcium Caseinate | 3.00 |
| 14 | Whey Protein Isolate | 3.00 |
| 15 | Peanuts | 7.98 |
| 16 | Soy Crisps | 0.00 |
| | Total | 100.00 |

Chocolate Peanut Butter
Formula # 5367-45-35
Serving size 55 g, 3 g alginate

| # | Ingredient | Percentage |
|---|---|---|
| 1 | Sugar-Free Choc Coating | 20.00 |
| 2 | High Fructose Corn Syrup | 15.00 |
| 3 | Glycerine | 6.00 |
| 4 | Peanut Butter | 10.00 |
| 5 | Peanut Butter Flavor | 2.00 |
| 6 | Maltodextrin | 5.00 |
| 7 | Inulin | 5.00 |
| 8 | Erythritol | 5.00 |
| 9 | Alginate | 5.45 |
| 10 | Peanut Flour | 3.40 |
| 11 | Dicalcium Phosphate Anyhdrous | 1.88 |
| 12 | Soy Protein Isolate | 4.00 |
| 13 | Calcium Caseinate | 4.00 |
| 14 | Whey Protein Isolate | 4.00 |
| 15 | Peanuts | 9.27 |
| | Total | 100.00 |

Chocolate Peanut Butter
Formula # 5367-45-01
Serving size 55 g, contains 200 mg Ca, 1 g alginate, 1 g pectin, 3 g inulin

| # | Ingredient | Percentage |
|---|---|---|
| 1 | Sugar-Free Choc Coating | 18.00 |
| 2 | Water | 12.50 |
| 3 | Glycerine | 9.00 |
| 4 | Peanut Butter | 10.00 |
| 8 | Peanut Butter Flavor | 1.00 |
| 11 | Inulin | 5.45 |
| 12 | Erythritol | 8.00 |
| 13 | Fructose | 3.50 |
| 15 | Alginate | 2.15 |
| 16 | Pectin | 3.04 |
| 17 | Soy Protein Isolate | 5.50 |
| 18 | Calcium Caseinate | 5.50 |
| 19 | Whey Protein Isolate | 8.00 |
| 20 | Tricalcium Phosphate | 0.96 |
| 21 | Peanut Flour | 7.40 |
| | Total | 100.00 |

Aw 0.686

Chocolate Peanut Butter
Formula # 5367-45-02
Serving size 55 g, contains 200 mg Ca, 1 g alginate, 1 g pectin, 3 g inulin

| # | Ingredient | Percentage |
|---|---|---|
| 1 | Sugar-Free Choc Coating | 15.00 |
| 2 | Water | 14.00 |
| 3 | Glycerine | 10.00 |
| 4 | Peanut Butter | 10.00 |
| 8 | Peanut Butter Flavor | 2.00 |
| 11 | Inulin | 5.45 |
| 12 | Erythritol | 7.00 |
| 15 | Alginate | 2.15 |
| 16 | Pectin | 3.04 |
| 21 | Peanut Flour | 5.00 |
| 20 | Tricalcium Phosphate | 0.96 |
| 17 | Soy Protein Isolate | 5.00 |
| 18 | Calcium Caseinate | 5.00 |
| 19 | Whey Protein Isolate | 7.50 |
| | Peanuts | 5.00 |
| | Soy Crisps | 2.90 |
| | Total | 100.00 |

Aw 0.726

Chocolate Peanut Butter
Formula # 5367-45-03
Serving size 55 g, contains 200 mg Ca, 1 g alginate, 1 g pectin, 3 g inulin

| # | Ingredient | Percentage |
|---|---|---|
| 1 | Sugar-Free Choc Coating | 15.00 |
| 2 | Water | 14.50 |
| 3 | Glycerine | 11.00 |
| 4 | Peanut Butter | 10.00 |
| 8 | Peanut Butter Flavor | 2.00 |
| 11 | Inulin | 5.45 |
| 12 | Erythritol | 10.00 |
| 15 | Alginate | 2.15 |
| 16 | Pectin | 3.04 |
| 21 | Peanut Flour | 3.40 |
| 20 | Tricalcium Phosphate | 0.96 |
| 17 | Soy Protein Isolate | 3.50 |
| 18 | Calcium Caseinate | 3.50 |
| 19 | Whey Protein Isolate | 3.50 |
| | Hydrolysed Whey | 3.50 |
| | Peanuts | 6.00 |
| | Soy Crisps | 2.50 |
| | Total | 100.00 |

A w 0.710

Chocolate Raspberry
Formula # 5367-44-01
Serving size 45 g, contains 200 mg Ca, 5% alginate/pectin mix, 3 g inulin

| # | Ingredient | Percentage |
|---|---|---|
| 1 | Chocolate Coating | 15.00 |
| 2 | HFCS | 10.00 |
| 3 | Water | 10.00 |
| 4 | Glycerine | 8.00 |
| 5 | Honey | 2.00 |
| 6 | Canola Oil | 6.00 |
| 7 | Raspberry Flavor | 1.00 |
| 8 | Raspberry Flavor | 0.50 |
| 9 | White Chocolate Flavor | 0.35 |
| 10 | Vanilla Flavor | 0.20 |
| 11 | Inulin | 6.70 |
| 12 | Erythritol | 5.00 |
| 13 | Malic Acid | 0.25 |
| 14 | Tricalcium Phosphate | 1.15 |
| 15 | Alginate Pectin Mix | 5.00 |
| 16 | Soy Protein Isolate | 7.00 |
| 17 | Calcium Caseinate | 6.00 |
| 18 | Whey Protein Isolate | 6.00 |
| 19 | Dried Raspberry | 4.85 |
| 20 | Soy Crisps, 80% protein | 5.00 |
| | Total | 100.00 |

Aw at 0.677

Chocolate Peanut Butter
Formula # 5367-45-04
Serving size 55 g, contains 200 mg Ca, 1 g alginate, 1 g pectin, 3 g inulin

| # | Ingredient | Percentage |
|---|---|---|
| 1 | Sugar-Free Choc Coating | 20.00 |
| 2 | Water | 14.50 |
| 3 | Glycerine | 12.00 |
| 4 | Peanut Butter | 10.00 |
| 5 | Peanut Butter Flavor | 2.00 |
| 6 | Inulin | 5.45 |
| 7 | Erythritol | 6.00 |
| 8 | Alginate | 2.15 |
| 9 | Pectin | 3.04 |
| 10 | Peanut Flour | 3.40 |
| 11 | Tricalcium Phosphate | 0.96 |
| 12 | Soy Protein Isolate | 4.00 |
| 13 | Calcium Caseinate | 4.00 |
| 14 | Whey Protein Isolate | 4.00 |
| 16 | Peanuts | 6.00 |
| 17 | Soy Crisps | 2.50 |
| | Total | 100.00 |

Aw 0.698

Chocolate Raspberry
Formula # 5367-44-02
Serving size 45 g, contains 200 mg Ca, 5% alginate/pectin mix, 3 g inulin

| # | Ingredient | Percentage |
|---|---|---|
| 1 | Chocolate Coating | 15.00 |
| 2 | HFCS | 10.50 |
| 3 | Water | 10.50 |
| 4 | Glycerine | 8.00 |
| 5 | Honey | 2.00 |
| 6 | Canola Oil | 6.00 |
| 7 | Raspberry Flavor | 1.00 |
| 8 | Raspberry Flavor | 0.50 |
| 9 | White Chocolate Flavor | 0.35 |
| 10 | Vanilla Flavor | 0.50 |
| 11 | Inulin | 6.70 |
| 12 | Erythritol | 2.00 |
| 13 | Malic Acid | 0.25 |
| 14 | Alginate | 2.50 |
| 15 | Pectin | 2.50 |
| 16 | Soy Protein Isolate | 7.00 |
| 17 | Calcium Caseinate | 6.00 |
| 18 | Whey Protein Isolate | 6.00 |
| 19 | Tricalcium Phosphate | 1.15 |
| 20 | Dried Raspberry | 6.00 |
| 21 | Soy Crisps, 80% protein | 5.55 |
| | Total | 100.00 |

Chocolate Peanut Butter
Formula # 5367-45-31
Serving size 55 g, 5 g alginate

| # | Ingredient | Percentage |
|---|---|---|
| 1 | Sugar-Free Choc Coating | 18.20 |
| 2 | HFCS | 12.00 |
| 3 | Maltitol | 12.00 |
| 4 | Glycerine | 3.00 |
| 5 | Peanut Butter | 3.00 |
| 6 | Peanut Butter Flavor | 3.00 |
| 7 | Vanilla | 0.50 |
| | Mix for 0.5 min | |
| 8 | Alginate | 9.10 |
| | Mix for 1 min | |
| 9 | Erythritol | 8.00 |
| 10 | Inulin | 5.00 |
| 11 | Peanut Flour | 5.00 |
| 12 | Hydrolyzed Whey Isolate | 4.00 |
| 13 | Sucrolose | 0.01 |
| | Mix for 0.5 min | |
| 14 | Tricalcium Phosphate | 0.00 |
| 15 | Calcium Carbonate | 0.00 |
| 16 | Whey Protein Isolate | 8.50 |
| 17 | Peanuts | 8.69 |
| | Mix for 1 min | |
| | Total | 100.00 |

Aw = 0.52

Chocolate Peanut Butter
Formula # 5367-45-05
Serving size 55 g, contains 200 mg Ca, 1 g alginate, 1 g pectin, 3 g inulin
GHB

| # | Ingredient | Percentage |
|---|---|---|
| 1 | Sugar-Free Choc Coating | 20.00 |
| 2 | Water | 14.50 |
| 3 | Glycerine | 12.00 |
| 4 | Peanut Butter | 10.00 |
| 5 | Peanut Butter Flavor | 2.00 |
| 6 | Inulin | 5.45 |
| 7 | Erythritol | 6.00 |
| 8 | Alginate | 4.30 |
| 9 | Pectin | 0.00 |
| 10 | Peanut Flour | 3.40 |
| 11 | Tricalcium Phosphate | 0.96 |
| 12 | Soy Protein Isolate | 4.00 |
| 13 | Calcium Caseinate | 4.00 |
| 14 | Whey Protein Isolate | 4.00 |
| 16 | Peanuts | 6.00 |
| 17 | Soy Crisps | 2.50 |
| | Total | 99.11 |

Aw 0.713

Chocolate Peanut Butter
Formula # 5367-45-06
Serving size 55 g, contains 200 mg Ca, 1 g alginate, 1 g pectin, 3 g inulin

| # | Ingredient | Percentage |
|---|---|---|
| 1 | Sugar-Free Choc Coating | 20.00 |
| 2 | Water | 14.50 |
| 3 | Glycerine | 12.00 |
| 4 | Peanut Butter | 10.00 |
| 5 | Peanut Butter Flavor | 2.00 |
| 6 | Inulin | 7.00 |
| 7 | Erythritol | 6.00 |
| 8 | Alginate | 0.00 |
| 9 | Pectin | 0.00 |
| 10 | Peanut Flour | 3.40 |
| 11 | Tricalcium Phosphate | 0.00 |
| 12 | Soy Protein Isolate | 4.00 |
| 13 | Calcium Caseinate | 4.00 |
| 14 | Whey Protein Isolate | 4.00 |
| 16 | Peanuts | 6.00 |
| 17 | Soy Crisps | 2.50 |
| | MonoCalcium Phosphate | 4.60 |
| | Total | 100.00 |

a = 0.705

Chocolate Peanut Butter
Formula # 5367-45-07
Serving size 55 g, contains 200 mg Ca, 1 g alginate, 1 g pectin, 3 g inulin

| # | Ingredient | Percentage |
|---|---|---|
| 1 | Sugar-Free Choc Coating | 20.00 |
| 2 | Water | 14.50 |
| 3 | Glycerine | 12.00 |
| 4 | Peanut Butter | 10.00 |
| 5 | Peanut Butter Flavor | 2.00 |
| 6 | Inulin | 6.10 |
| 7 | Erythritol | 6.00 |
| 8 | Alginate | 0.00 |
| 9 | Pectin | 0.00 |
| 10 | Peanut Flour | 3.40 |
| 11 | Tricalcium Phosphate | 0.00 |
| 12 | Soy Protein Isolate | 4.00 |
| 13 | Calcium Caseinate | 4.00 |
| 14 | Whey Protein Isolate | 4.00 |
| 16 | Peanuts | 6.00 |
| 17 | Soy Crisps | 2.50 |
| | Calcium Lactate | 5.50 |
| | Total | 100.00 |

Aw 0.690

Chocolate Peanut Butter, Dec. 22, 2003
Formula # 5367-45-08
Serving size 55 g, contains 300 mg Ca, 1 g alginate, 1 g pectin, 3 g inulin

| # | Ingredient | Percentage |
|---|---|---|
| 1 | Sugar-Free Choc Coating | 20.00 |
| 2 | Water | 14.50 |
| 3 | Glycerine | 12.00 |
| 4 | Peanut Butter | 10.00 |
| 5 | Peanut Butter Flavor | 2.00 |
| 6 | Inulin | 5.45 |
| 7 | Erythritol | 2.76 |
| 8 | Alginate | 2.15 |
| 9 | Pectin | 3.04 |
| 10 | Peanut Flour | 3.40 |
| 11 | Tricalcium Phosphate | 0.00 |
| 12 | Soy Protein Isolate | 4.00 |
| 13 | Calcium Caseinate | 4.00 |
| 14 | Whey Protein Isolate | 4.00 |
| 16 | Peanuts | 6.00 |
| 17 | Soy Crisps | 2.50 |
| | Calcium Lactate | 4.20 |
| | Total | 100.00 |

Chocolate Peanut Butter,
Formula # 5367-45-09
Serving size 55 g, contains 300 mg Ca, 3 g inulin

| # | Ingredient | Percentage |
|---|---|---|
| 1 | Sugar-Free Choc Coating | 20.00 |
| 2 | Water | 14.50 |
| 3 | Glycerine | 12.00 |
| 4 | Peanut Butter | 10.00 |
| 5 | Peanut Butter Flavor | 2.00 |
| 6 | Inulin | 5.45 |
| 7 | Erythritol | 5.52 |
| 8 | Alginate | 0.00 |
| 9 | Pectin | 0.00 |
| 10 | Peanut Flour | 8.59 |
| 11 | Tricalcium Phosphate | 1.44 |
| 12 | Soy Protein Isolate | 4.00 |
| 13 | Calcium Caseinate | 4.00 |
| 14 | Whey Protein Isolate | 4.00 |
| 16 | Peanuts | 6.00 |
| 17 | Soy Crisps | 2.50 |
| | Calcium Lactate | 0.00 |
| | Total | 100.00 |

Chocolate Peanut Butter
Formula # 5367-45-10
Serving size 55 g, contains 200 mg Ca, 3 g inulin
Test layer bar

| # | Ingredient | Percentage |
|---|---|---|
| 1 | Sugar-Free Choc Coating | 15.00 |
| | Caramel | 13.62 |
| 8 | Alginate | 4.30 |
| 9 | Pectin | 6.08 |
| | Water | 10.00 |
| | Glycerine | 10.00 |
| 16 | Peanuts | 6.00 |
| 2 | Water | 4.00 |
| 3 | Glycerine | 3.00 |
| 4 | Peanut Butter | 4.00 |
| 5 | Peanut Butter Flavor | 1.00 |
| 6 | Inulin | 15.57 |
| 10 | Peanut Flour | 2.00 |
| 11 | Tricalcium Phosphate | 2.74 |
| 13 | Calcium Caseinate | 2.69 |
| | Total | 100.00 |

Chocolate Peanut Butter
Formula # 5367-45-11
Serving size 55 g
Test layer bar

| # | Ingredient | Percentage |
|---|---|---|
| 1 | Sugar-Free Choc Coating | 15.00 |
| | Caramel | 13.62 |
| 8 | Alginate | 4.30 |
| 9 | Pectin | 6.08 |
| | Water | 0.00 |
| | Glycerine | 10.00 |
| 16 | Peanuts | 6.00 |
| 2 | Water | 4.00 |
| 3 | Glycerine | 3.00 |
| 4 | Peanut Butter | 4.00 |
| 5 | Peanut Butter Flavor | 1.00 |
| 6 | Inulin | 15.57 |
| 10 | Peanut Flour | 2.00 |
| 11 | Tricalcium Phosphate | 2.74 |
| 13 | Calcium Caseinate | 2.69 |
| | Total | 90.00 |

Chocolate Peanut Butter
Formula # 5367-45-12
Serving size 55 g, contains 300 mg Ca

| # | Ingredient | Percentage |
|---|---|---|
| 1 | Sugar-Free Choc Coating | 20.00 |
| 2 | Water | 14.50 |
| 3 | Glycerine | 12.00 |
| 4 | Peanut Butter | 10.00 |
| 5 | Peanut Butter Flavor | 2.00 |
| 6 | Inulin | 5.45 |
| 7 | Erythritol | 2.76 |
| 8 | Alginate | 5.19 |
| 9 | Pectin | 0.00 |
| 10 | Peanut Flour | 3.40 |
| 11 | Tricalcium Phosphate | 0.00 |
| 12 | Soy Protein Isolate | 4.00 |
| 13 | Calcium Caseinate | 4.00 |
| 14 | Whey Protein Isolate | 4.00 |
| 16 | Peanuts | 6.00 |
| 17 | Soy Crisps | 2.50 |
| | Calcium Lactate | 4.20 |
| | Total | 100.00 |

What is claimed is:

1. A formed food comprising an effective amount of at least one soluble anionic fiber and an effective amount of at least one multivalent cation wherein the combination of fiber and cation increases viscosity of digesta in an animal.

2. A formed food of claim 1, wherein the formed food product is produced by a process selected from the group consisting of extrusion, pressing, molding, wire cutting, and mixtures thereof.

3. A formed food of claim 1, wherein the at least one soluble anionic fiber comprises alginate and pectin.

4. A formed food of claim 3, wherein the alginate comprises an intermediate molecular weight form of alginate and a low molecular weight form of alginate.

5. A formed food of claim 3, wherein total alginate to total pectin is from about 8:1 to about 1:8.

6. A formed food of claim 1, wherein the multivalent cation is selected from the group consisting of calcium, magnesium, aluminum, manganese, iron, nickel, copper, zinc, strontium, barium, bismuth, chromium, vanadium, and lanthanum, their salts and mixtures thereof.

7. A formed food of claim 6, wherein the multivalent cation salt is selected from the group of multivalent cation salts consisting of formate, fumarate, acetate, propionate, butyrate, caprylate, valerate, lactate, citrate, malate and gluconate, citrate malate, chloride, potassium, and mixtures thereof.

8. A formed food of claim 6, wherein the multivalent cation is calcium and wherein the salt is selected from the group consisting of calcium citrate, calcium tartrate, calcium succinate, calcium fumarate, calcium adipate, calcium malate, calcium lactate, calcium gluconate, dicalcium phosphate dihydrate, anhydrous calcium diphosphate, calcium citrate malate, dicalcium phosphate anhydrous, calcium chloride, calcium acetate monohydrate, and mixtures thereof.

9. A formed food of claim 1, wherein a ratio of the at least two soluble anionic fibers to the at least one multivalent cation to in the ingestible composition is from about 20:1 to about 7:1.

10. An ingestible composition comprising:
a solid phase comprising at least one soluble anionic fiber in a total amount of from about 0.5 g to about 10 g per serving and
a fluid phase in intimate contact with the solid phase, the fluid phase comprising at least one multivalent cation in an amount of from about 50 to about 300 mg of elemental multivalent cation per serving.

11. An ingestible composition in claim 10, wherein the solid phase is a baked dough.

12. An ingestible composition in claim 10, wherein the fluid phase in not baked

13. An ingestible composition of claim 12, wherein the fluid phase is selected from the group consisting of jam, jelly, pudding, custard, frosting, icing, soft center and coating material.

14. An ingestible composition of claim 10, wherein the fluid phase is within the solid phase.

15. An ingestible composition of claim 10, wherein the fluid phase is deposited on the solid phase.

16. An ingestible composition of claim 10, wherein the at least one soluble anionic fiber comprises alginate and pectin.

17. An ingestible composition of claim 16, wherein the alginate comprises an intermediate molecular weight form of alginate and a low molecular weight form of alginate.

18. An ingestible composition of claim 16, wherein total alginate to total pectin is from about 8:1 to about 1:8.

19. An ingestible composition of claim 10, wherein at least one multivalent cation is a salt selected from the group consisting of formate, fumarate, acetate, propionate, butyrate, caprylate, valerate, lactate, citrate, malate and gluconate, citrate malate, chloride, phosphate and mixtures thereof.

20. An ingestible composition of claim 10, wherein the multivalent cation is calcium and wherein the salt is selected from the group consisting of calcium citrate, calcium tartrate, calcium succinate, calcium fumarate, calcium adipate, calcium malate, calcium lactate, calcium gluconate, dicalcium phosphate dihydrate, anhydrous calcium diphosphate, dicalcium phosphate anhydrous, calcium chloride, calcium acetate monohydrate, and mixtures thereof.

21. An ingestible composition of claim 10, wherein a ratio of the at least one soluble anionic fiber to the at least one multivalent cation in the ingestible composition is from about 20:1 to about 7:1.

22. A method for inducing satiety in an animal, the method comprising the step of orally administering to the animal a serving of a formed food comprising at least one soluble anionic fiber and at least one multivalent cation.

23. A method for inducing satiety in an animal, the method comprising the step of orally administering to the animal a serving of a food comprising
a formed solid phase comprising at least one soluble anionic fiber in a total amount of from about 0.5 g to about 10 g per serving and
a fluid phase in intimate contact with the formed solid phase, the fluid phase comprising calcium in an amount of from about 50 to about 500 mg of elemental calcium per serving.

24. A method for reducing caloric intake in an animal, the method comprising the step of administering to the animal a serving of a formed food comprising at least one soluble anionic fibers and at least one multivalent cation.

25. A method for reducing caloric intake in an animal, the method comprising the step of orally administering to the animal a serving of an food comprising
a formed solid phase comprising alginate and pectin in a total amount of from about 0.5 g to about 10 g per serving and
a fluid phase in intimate contact with the formed solid phase, the fluid phase comprising calcium in an amount of from about 50 to about 300 mg of elemental calcium per serving.

26. A method for reducing weight in an animal, the method comprising the step of orally administering to the animal a serving of a formed food comprises at least one soluble anionic fiber and at least one multivalent cation.

27. A method for reducing weight in an animal, the method comprising the step of orally administering to the animal a serving of an ingestible composition comprising
a formed solid phase comprising at least one soluble anionic fiber in a total amount of from about 0.5 g to about 10 g per serving and
a fluid phase in intimate contact with the formed solid phase, the fluid phase comprising calcium in an amount of from about 50 to about 300 mg of elemental calcium per serving.

28. A method for improving weight reduction by at least 5% in an animal, the method comprising the step of orally administering to the animal a serving of a formed food comprising at least one soluble anionic fiber and a multivalent cation, wherein the weight reduction improvement is measured after four months of daily administration of the ingestible composition.

29. A method for improving weight reduction of claim 28, wherein the weight reduction is selected from the group consisting of at least about 10%.

30. A method for improving weight reduction by at least 5% in an animal, the method comprising the step of orally administering to the animal an ingestible composition comprising.
a formed solid phase comprising alginate and pectin in a total amount of from about 0.5 g to about 10 g per serving and
a fluid phase in intimate contact with the solid phase, the fluid phase comprising calcium in an amount of from about 50 to about 300 mg of elemental calcium per serving, wherein the weight reduction improvement is measured after four months of daily administration of the ingestible composition.

31. An ingestible composition comprising:
a formed solid phase comprising at least one soluble anionic fiber in a total amount of from about 0.5 g to about 10 g per serving and
a fluid phase in intimate contact with the baked phase, the fluid phase comprising calcium in an amount of from about 50 to about 300 mg of elemental calcium per serving.

32. An ingestible composition of claim 31, wherein the formed solid phase is extruded, molded, wire-cut or pressed.

33. An ingestible composition of claim 31, wherein the formed solid phase is baked.

34. An ingestible composition of claim 31, wherein the fluid phase is selected from the group consisting of jam, jelly, pudding, custard, frosting, icing, soft center and coating material.

35. An ingestible composition of claim 31, wherein the fluid phase is within the formed solid phase.

36. An ingestible composition of claim 31, wherein the fluid phase is deposited on the formed solid phase.

37. An ingestible composition of claim 31, wherein the least one soluble anionic fiber comprises alginate and pectin.

38. An ingestible composition of claim 37, wherein the alginate comprises an intermediate molecular weight form of alginate and a low molecular weight form of alginate.

39. An ingestible composition of claim 37, wherein total alginate to total pectin is from about 8:1 to about 1:8.

40. An ingestible composition of claim 31, wherein calcium is a salt and the calcium salt is selected from the group consisting of calcium citrate, calcium tartrate, calcium succinate, calcium fumarate, calcium adipate, calcium malate, calcium lactate, calcium gluconate, dicalcium phosphate dihydrate, anhydrous calcium diphosphate, dicalcium phosphate anhydrous, calcium chloride, calcium acetate monohydrate, and mixtures thereof.

41. An ingestible composition of claim 31, wherein a ratio of the at least one soluble anionic fiber to elemental calcium to in the ingestible composition is from about 20:1 to about 7:1.
